(19) United States
(12) Patent Application Publication
(10) Pub. No.: US 2007/0021500 A1
(43) Pub. Date: Jan. 25, 2007

(54) Methods for neuroprotection

(76) Inventors: Roy E. Twyman, Doylestown, PA (US); Boyu Zhao, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/481,601

(22) Filed: Jul. 6, 2006

Related U.S. Application Data
(60) Provisional application No. 60/698,403, filed on Jul. 12, 2005.

(51) Int. Cl. A61K 31/325

(52) U.S. Cl. 514/483; 514/489

(57) ABSTRACT

This invention is directed to methods for providing neuroprotection comprising administering to a subject in need thereof a therapeutically effective amount of a compound selected from the group consisting of Formula (I) and Formula (II), or a pharmaceutically acceptable salt or ester thereof: 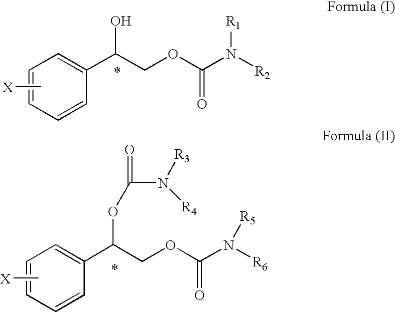
wherein phenyl is substituted at X with one to five halogen atoms selected from the group consisting of fluorine, chlorine, bromine and iodine; and, R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

### BACKGROUND OF THE INVENTION

1. Field of the Invention

The present invention relates generally to the fields of pharmacology, neurology and psychiatry and to methods of protecting the cells of a mammalian central nervous system from damage or injury. More specifically, this invention provides methods for the use of certain carbamate compounds for neuroprotection.

2. Description of the Related Art

Injuries or trauma of various kinds to the central nervous system (CNS) or the peripheral nervous system (PNS) can produce profound and long-lasting neurological and/or psychiatric symptoms and disorders. One form that this can take is the progressive death of neurons or other cells of the central nervous system (CNS), i.e., neurodegeneration or neuronal degeneration. Neuronal degeneration as a result of, for example; Alzheimer's disease, multiple sclerosis, cerebral-vascular accidents (CVAs) stroke, traumatic brain injury, spinal cord injuries, degeneration of the optic nerve, e.g., ischemic optic neuropathy or retinal degeneration and other central nervous system disorders is an enormous medical and public health problem by virtue of both its high incidence and the frequency of long-term sequelae. Animal studies and clinical trials have shown that amino acid transmitters (especially glutamate), oxidative stress and inflammatory reactions contribute strongly to cell death in these conditions.

Upon injury or upon ischemic insult, damaged neurons release massive amounts of the neurotransmitter glutamate, which is excitotoxic to the surrounding neurons (Choi et al., (1988), Neuron 1: 623-634; Rothman et al., (1984), J. Neurosci. 4: 1884-1891; Choi end Rothman, (1990), Ann. Rev. Neurosci. 13: 171-182; David et al., (1988), Exp. Eye Res. 46:657-662; Drejer et al., (1985), J. Neurosci. 45:145-151. Glutamate is a negatively charged amino acid that is an excitatory synaptic transmitter in the mammalian nervous system. Although the concentration of glutamate can reach the millimolar range in nerve terminals its extracellular concentration is maintained at a low level to prevent neurotoxicity. It has been noted that glutamate can be toxic to neurons if presented at a high concentration. The term “excitotoxicity” has been used to describe the cytotoxic effect that glutamate (and other such excitatory amino acids) can have on neurons when applied at high dosages.

Physiologically, excessive release, inhibition of uptake, or both can achieve high levels of glutamate. Normally, a low concentration of extracellular glutamate is maintained by both neurons and astrocytes. Neurons store glutamate in intracellular stores and regulate its release. See, Reagan, R. F., Excitotoxicity and Central Nervous System Trauma, in The Neurobiology of Central Nervous Trauma, New York, Oxford University Press, 1994, pp. 173-181 (Salzman S K, Faden A I, eds). Astrocytes take up extracellular glutamate by specific transporters and convert the glutamate into glutamine that is then released for neuronal uptake. See, Robinson, M. B. & Dowd L A, Adv Pharmacol, 1997; 37:69-115. In the process of excitotoxicity, glutamate is released in a self-perpetuating manner by the neurons, resulting in excessive or prolonged activation of glutamate receptors.

The conjunction of such excessive glutamate stimulation on the energy-depleted neurons taken with the compromised ability of the neurosupportive astrocytes to sequester toxic levels of extracellular glutamate leads to neuronal death via necrosis and apoptosis. Various interventions are currently being examined to reduce neuronal death associated with central nervous system injuries and diseases. See, Kermer et al., Cell Tissue Res 298:383-395, 1999. Such therapies include glutamate release inhibitors, glutamate receptor antagonists, Ca2+ channel blockers, GABA receptor agonists, gangliosides, neurotrophic factors, calpain inhibitors, caspase inhibitors, free radical scavengers, immuno- and cell metabolism modulators.

For example, several studies have shown the involvement of glutamate in the pathophysiology of: 1) Huntington's disease (HD) (Coyle and Schwartz, (1976), Nature 263: 244-246; 2) Alzheimer's disease (AD) (Maragos et al, (1987), TINS 10: 65-68; 3) Epilepsy (Nadler et al, (1978), Nature 271: 676-677); 4) Lathyrism (Spencer et al, (1986), Lancet 239: 1066-1067; 5) Amyotropic Lateral Sclerosis (ALS) and Parkinsonian dementia of Guam (Caine et al, (1986), Lancet 2: 1067-1070) as well as in the neuropathology associated with stroke, ischemia and reperfusion (See, Dykens et al, (1987), J. Neurochem. 49: 1222-1228).

Thus, injury to neurons may be caused by overstimulation of receptors by excitatory amino acids including glutamate and aspartate (See, Lipton et al. (1994) New Engl. J. Med. 330:613 621). Indeed, the N-methyl-D-aspartate (NMDA) subtype of glutamate receptor is suggested to have many important roles in normal brain function, including synaptic transmission, learning and memory, and neuronal development (See, Lipston et al. (1994) supra; Meldrum et al. (1990) Trends Pharm. Sci. 11:379-387). However, over-stimulation of the NMDA subtype of glutamate receptor leads to increased free radical production and neuronal cell death, which can be modulated by antioxidants (See, Herin et al. (2001) J. Neurochem. 78:1307-1314; Rossato et al. (2002) Neurosci. Lett. 318:137-140).

In addition, in many chronic neurodegenerative conditions, inflammation and oxidative stress are key components of the pathology. These conditions include Alzheimer's disease (AD). Alzheimer's disease (AD) is characterized by the accumulation of neurofibrillary tangles and senile plaques, and a widespread, progressive degeneration of neurons in the brain. Senile plaques are rich in amyloid precursor protein (APP) that is encoded by the APP gene located on chromosome 21. A commonly accepted hypothesis underlying pathogenesis of AD is that abnormal proteolytic cleavage of APP leads to an excess extracellular accumulation of beta-amyloid (Aβ) peptide that has been shown to be toxic to neurons (See, Selkoe et al., (1996), J. Biol. Chem. 271: 487-498; Quinn et al., (2001), Exp. Neurol. 168: 203-212; Mattson et al., (1997), Alzheimer's Dis. Rev. 12: 1-14; Fakuyama et al., (1994), Brain Res. 667: 269-272).

Parkinson's disease (PD) is a progressive neurodegenerative disorder characterized by a dysfunction of movement consisting of akinesia, rigidity, tremor and postural abnormalities. This disease has been associated with the loss of nigro-striatal dopaminergic neuronal integrity and functionality as evidenced by substantial loss of dopaminergic neurons in substantia nigra pars compacta (SNpc) (See, Pakkenberg et al. (1991) J. Neurol. Neurosurg. Psychiat. 54:30-33), and a decrease in content, synaptic and vesicular transporters of dopamine in the striatum (see, for example, Guttnan et al. (1997) Neurology 48:1578-1583).

Death of neurons and supporting cells in the central (CNS) or peripheral (PNS) nervous system of mammals including humans as a result of trauma, injury of many kinds, ischemia, metabolic derangements, e.g., diabetes hypoxia, toxins or surgical intervention causes both acute and chronic and progressive loss of function and disability. Thus there is a need for the development of methods and compounds that can protect the cells of the mammalian nervous system from this degeneration, i.e., are neuroprotective.

### SUMMARY OF THE INVENTION

The present invention relates in general to neuroprotective methods, and more specifically to methods and compounds for prevention of damage to cells of the mammalian central and peripheral nervous system resulting from injury, trauma, surgery or acute or chronic disease processes.

This invention is based, in part, on the discovery that the administration of one or more members of a family of carbamate compounds either alone or in combination with one or more other neuroprotective medications provides a neuroprotective effect on the mammalian nervous system.

Neuroprotection provided by this invention includes protection from damage resulting from neural injury or insult and from neurotoxicity, including excitotoxicity. Thus, neuroprotection provided by this invention will be useful in the treatment of acute and chronic neurodegenerative disorders that may involve excitotoxicity, for example glutamate excitotoxicity, including stroke/ischemia, surgical trauma, Traumatic Brain Injury (TBI), blunt, closed or penetrating head trauma, epilepsy, Huntington's disease, Amyotrophic Lateral Sclerosis (ALS), diabetic neuropathy and hypoglycemic encephalopathy.

Neuroprotection provided by this invention may be brought about upon injured or diseased tissue or in a preventative fashion during or prior to events expected to lead to a neural insult.

The invention provides methods for providing neuroprotection; for inhibiting cell degeneration or cell death; for treatment or prophylaxis of a neurodegenerative disease; or for ameliorating the cytotoxic effect of a compound (for example, a excitatory amino acid such as glutamate; a toxin; or a prophylactic or therapeutic compound that exerts a cytotoxic side effect) in a subject in need thereof, by administering to the subject an effective amount of a compound of the invention, or it's pharmaceutically acceptable salt or ester either alone or in combination with another medication along with a pharmaceutically acceptable excipient. In various embodiments, the methods of the invention include protection against excitotoxicity, for example glutamate excitotoxicity.

In various embodiments, the subject, for example, a human, may be suffering from neural insult or injury; or may be suffering from a condition selected from substance abuse, trauma, stroke, ischemia, Huntington's disease, Alzheimer's disease, Parkinson's disease, prion disease, variant Creutzfeld-Jakob disease, amyotrophic or hypoglycemic encephalopathy; or may be undergoing surgery or other intervention. The subject may have a pre-existing condition that would benefit by neuroprotection or the patient may be treated to reduce deleterious effects of a concomitant or subsequent neural injury, such as may occur during surgery or other intervention.

Accordingly, the present invention provides methods for providing neuroprotection comprising administering to a subject in need thereof a therapeutically effective amount of a composition that comprises at least one compound having Formula 1 or Formula 2: 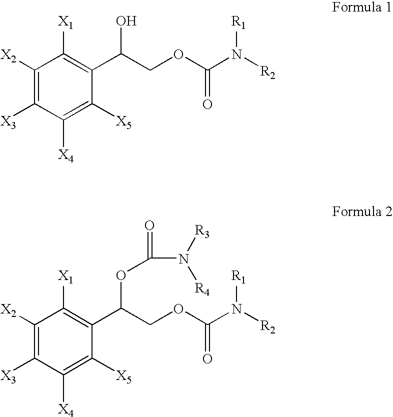
wherein R₁, R₂, R₃, and R₄ are, independently, hydrogen or C₁-C₄ alkyl; and X₁, X₂, X₃, X₄, and X₅ are, independently, hydrogen, fluorine, chlorine, bromine or iodine. The said C₁-C₄ alkyl group of Formula 1 or Formula 2 can be substituted or unsubstituted. In one aspect of the present invention, the C₁-C₄ alkyl group is substituted with a phenyl group. The phenyl group can be unsubstituted or substituted. In certain embodiments, the phenyl group is unsubstituted or substituted with halogen, C₁-C₄ alkyl, C₁-C₄ alkoxy, amino, nitro, or cyano.

In the present invention, X₁, X₂, X₃, X₄, and X₅ can be hydrogen, fluorine, chlorine, bromine or iodine. In certain embodiments, X₁, X₂, X₃, X₄, and X₅ are, independently, hydrogen or chlorine. In a preferred embodiment of the present invention, X₁ is fluorine, chlorine, bromine or iodine. In one aspect, X₁ is chlorine and X₂, X₃, X₄, and X₅ are, independently, hydrogen. In another preferred embodiment, R₁, R₂, R₃, and R₄ are, independently, hydrogen.

The present invention provides enantiomers of Formula 1 or Formula 2 for providing neuroprotection in a subject. In certain embodiments, a compound of Formula 1 or Formula 2 will be in the form of a single enantiomer thereof. In other embodiments, a compound of Formula 1 or Formula 2 will be in the form of an enantiomeric mixture in which one enantiomer predominates with respect to another enantiomer. In one aspect, the enantiomer will predominate to the extent of 90% or greater or to the extent of 98% or greater.

The present invention also provides methods comprising administering to a subject a neuroprotective amount of a composition that comprises at least one compound having Formula 1 or Formula 2 wherein R₁, R₂, R₃, and R₄ are, independently, hydrogen or C₁-C₄ alkyl; and X₁, X₂, X₃, X₄, and X₅ are, independently, hydrogen, fluorine, chlorine, bromine or iodine. In one embodiment, before administration of the composition to the subject, a determination will be made as to whether or not the subject suffers from some form of acute or chronic neurodegeneration or nervous system injury.

The present invention also provides methods comprising identifying a patient at risk of developing acute or chronic neurodegeneration or nervous system injury or a patient in need of treatment with a neuroprotective drug (NPD), as defined below and administering a composition that comprises at least one compound having Formula 1 or Formula 2 to the subject.

In certain embodiments of the present invention, a therapeutically effective amount of a compound having Formula 1 or Formula 2 for providing neuroprotection is from about 0.01 mg/Kg/dose to about 150 mg/Kg/dose.

In certain embodiments a therapeutically effective amount of pharmaceutical composition for providing neuroprotection comprising one or more of the enantiomers of this invention or a pharmaceutically acceptable salt or ester thereof and a pharmaceutically acceptable carrier or excipient is administered to a subject or patient in need of treatment with a neuroprotective drug or NPD.

Pharmaceutical compositions comprising at least one compound having Formula 1 or Formula 2 are administered to subjects in need thereof. In certain embodiments, a subject or patient in need of treatment with a neuroprotective drug or NPD may be one who has experienced some form of acute trauma or injury to the cells of the central or peripheral nervous or who has some form of acute or chronic neurodegenerative disorder. In one aspect, the subject or patient will be determined to be at risk for developing an acute or chronic neurodegenerative disorder at the time of administration, i.e., a patient in need of treatment with a neuroprotective drug. In other embodiments, a subject in need thereof is one who has acute injury or trauma to the cells of their nervous system at the time of administration.

### BRIEF DESCRIPTION OF THE FIGURES

FIG. 1: is a graph that shows the effects of increasing doses of TC on the number of neurons in different areas of the hippocampus counted at 14 days after li-pilo SE. Values are expressed as the number of neuronal cell bodies in each area of interest±S.E.M.

FIG. 2: is a graph that shows the effects of increasing doses of TC on the number of neurons in different nuclei of the amygdala counted at 14 days after li-pilo SE. Values are expressed as the number of neuronal cell bodies in each area of interest±S.E.M.

FIG. 3: is a graph that shows the effects of increasing doses of TC on the number of neurons in different nuclei of the thalamus counted at 14 days after li-pilo SE. Values are expressed as the number of neuronal cell bodies in each area of interest±S.E.M.

FIG. 4: is a graph that shows the effects of increasing doses of TC on the number of neurons in different areas of the cortex counted at 14 days after li-pilo SE. Values are expressed as the number of neuronal cell bodies in each area of interest±S.E.M.

FIG. 5: is a graph that shows the effects of increasing doses of TC on the latency to the first spontaneous seizure. Values are expressed as the mean latency in days for each group±S.E.M.

FIG. 6: is a graph that shows the effects of increasing doses of TC on the frequency of spontaneous seizures video-recorded over a 4 weeks period. Values are expressed as the mean number of seizures±S.E.M. The total represents the total number of seizures observed during the 4 weeks of video-recording and the mean represents the mean number of seizures per week. The Anova test demonstrated an effect of the treatment on the total number of seizures (p=0.045) and the mean number of seizures per week (p=0.045)

FIG. 7: shows the total number of seizures video-recorded over four weeks plotted according to the latency to the first spontaneous seizure (SL=short latency, LL=long latency). Values are expressed as the mean number of seizures for each subgroup±S.E.M. The ANOVA test did not show any significant effect of the treatment.

FIG. 8: shows the correlation between the latency to the first spontaneous seizure and the total number of seizures observed during the four following weeks.

### DETAILED DESCRIPTION OF THE INVENTION

The Carbamate Compounds of the Invention

The present invention provides methods of using 2-phenyl-1,2-ethanediol monocarbomates and dicarbamates to Neuroprotection to patients in need thereof.

Suitable methods for synthesizing and purifying carbamate compounds, including carbamate enantiomers, used in the methods of the present invention are well known to those skilled in the art. For example, pure enantiomeric forms and enantiomeric mixtures of 2-phenyl-1,2-ethanediol monocarbomates and dicarbamates are described in U.S. Pat. Nos. 5,854,283, 5,698,588, and 6,103,759, the disclosures of which are herein incorporated by reference in their entirety.

Representative carbamate compounds according to the present invention include those having Formula 1 or Formula 2: 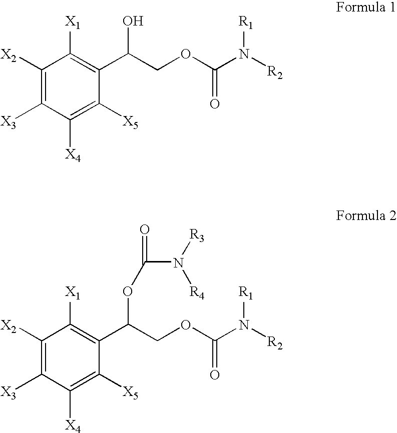
wherein R₁, R₂, R₃, and R₄ are, independently, hydrogen or C₁-C₄ alkyl and X₁, X₂, X₃, X₄, and X₅ are, independently, hydrogen, fluorine, chlorine, bromine or iodine.

“C₁-C₄ alkyl” as used herein refers to substituted or unsubstituted aliphatic hydrocarbons having from 1 to 4 carbon atoms. Specifically included within the definition of “alkyl” are those aliphatic hydrocarbons that are optionally substituted. In a preferred embodiment of the present invention, the C₁-C₄ alkyl is either unsubstituted or substituted with phenyl.

The term “phenyl”, as used herein, whether used alone or as part of another group, is defined as a substituted or unsubstituted aromatic hydrocarbon ring group having 6 carbon atoms. Specifically included within the definition of “phenyl” are those phenyl groups that are optionally substituted. For example, in a preferred embodiment of the present invention, the, “phenyl” group is either unsubstituted or substituted with halogen, C₁-C₄ alkyl, C₁-C₄ alkoxy, amino, nitro, or cyano.

In a preferred embodiment of the present invention, X₁ is fluorine, chlorine, bromine or iodine and X₂, X₃, X₄, and X₅ are hydrogen.

In another preferred embodiment of the present invention, X₁, X₂, X₃, X₄, and X₅ are, independently, chlorine or hydrogen.

In another preferred embodiment of the present invention, R₁, R₂, R₃, and R₄ are all hydrogen.

It is understood that substituents and substitution patterns on the compounds of the present invention can be selected by one of ordinary skill in the art to provide compounds that are chemically stable and that can be readily synthesized by techniques known in the art as well as the methods provided herein.

Representative 2-phenyl-1,2-ethanediol monocarbomates and dicarbamates include, for example, the following compounds:

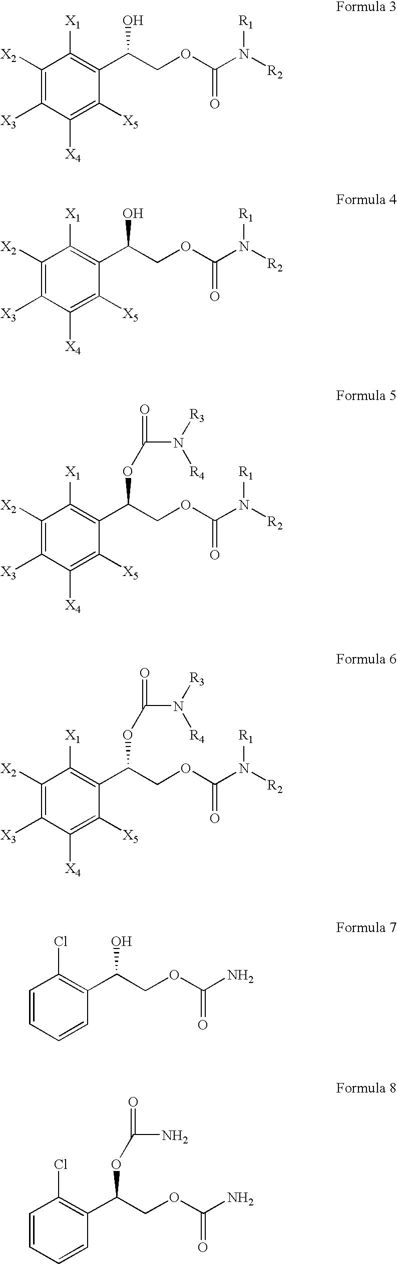

The present invention includes the use of isolated enantiomers of Formula 1 or Formula 2. In one preferred embodiment, a pharmaceutical composition comprising the isolated S-enantiomer of Formula 1 is used to provide neuroprotection in a subject. In another preferred embodiment, a pharmaceutical composition comprising the isolated R-enantiomer of Formula 2 is used to provide neuroprotection in a subject. In another embodiment, a pharmaceutical composition comprising the isolated S-enantiomer of Formula 1 and the isolated R-enantiomer of Formula 2 can be used to provide neuroprotection in a subject.

The present invention also includes the use of mixtures of enantiomers of Formula 1 or Formula 2. In one aspect of the present invention, one enantiomer will predominate. An enantiomer that predominates in the mixture is one that is present in the mixture in an amount greater than any of the other enantiomers present in the mixture, e.g., in an amount greater than 50%. In one aspect, one enantiomer will predominate to the extent of 90% or to the extent of 91%, 92%, 93%, 94%, 95%, 96%, 97% or 98% or greater. In one preferred embodiment, the enantiomer that predominates in a composition comprising a compound of Formula 1 is the S-enantiomer of Formula 1. In another preferred embodiment, the enantiomer that predominates in a composition comprising a compound of Formula 2 is the R-enantiomer of Formula 2.

In a preferred embodiment of the present invention, the enantiomer that is present as the sole enantiomer or as the predominate enantiomer in a composition of the present invention is represented by Formula 3 or Formula 5, wherein X₁, X₂, X₃, X₄, X₅, R₁, R₂, R₃, and R₄ are defined as above, or by Formula 7 or Formula 8.

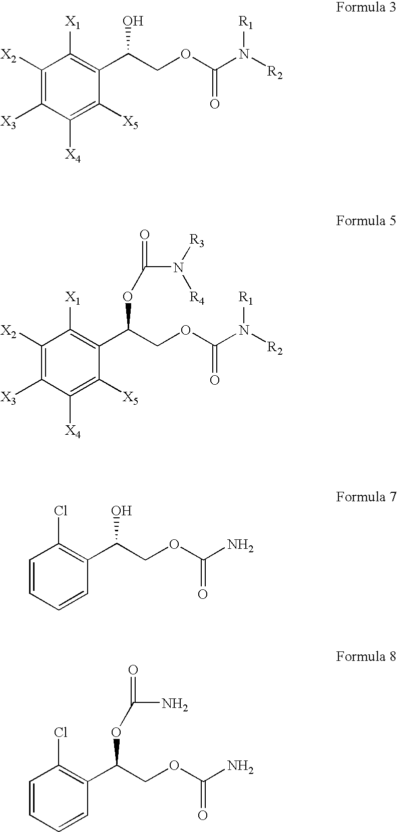

The present invention provides methods of using enantiomers and enantiomeric mixtures of compounds represented by Formula 1 and Formula 2. A carbamate enantiomer of Formula 1 or Formula 2 contains an asymmetric chiral carbon at the benzylic position, which is the aliphatic carbon adjacent to the phenyl ring.

An enantiomer that is isolated is one that is substantially free of the corresponding enantiomer. Thus, an isolated enantiomer refers to a compound that is separated via separation techniques or prepared free of the corresponding enantiomer. The term “substantially free,” as used herein, means that the compound is made up of a significantly greater proportion of one enantiomer. In preferred embodiments, the compound includes at least about 90% by weight of a preferred enantiomer. In other embodiments of the invention, the compound includes at least about 99% by weight of a preferred enantiomer. Preferred enantiomers can be isolated from racemic mixtures by any method known to those skilled in the art, including high performance liquid chromatography (HPLC) and the formation and crystallization of chiral salts, or preferred enantiomers can be prepared by methods described herein.

Methods for the preparation of preferred enantiomers would be known to one of skill in the art and are described, for example, in Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972).

Additionally, compounds of the present invention can be prepared as described in U.S. Pat. No. 3,265,728 (the disclosure of which is herein incorporated by reference in its entirety and for all purposes), U.S. Pat. No. 3,313,692 (the disclosure of which is herein incorporated by reference in its entirety and for all purposes), and the previously referenced U.S. Pat. Nos. 5,854,283, 5,698,588, and 6,103,759 (the disclosures of which are herein incorporated by reference in their entirety and for all purposes).

The Nature of Neuroprotection

Patients with injury or damage of any kind to the central (CNS) or peripheral (PNS) nervous system including the retina may benefit from these neuroprotective methods. This nervous system injury may take the form of an abrupt insult or an acute injury to the nervous system as in, for example, acute neurodegenerative disorders including, but not limited to; acute injury, hypoxia-ischemia or the combination thereof resulting in neuronal cell death or compromise. Acute injury includes, but is not limited to, Traumatic Brain Injury (TBI) including, closed, blunt or penetrating brain trauma, focal brain trauma, diffuse brain damage, spinal cord injury, intracranial or intravertebral lesions (including, but not limited to, contusion, penetration, shear, compression or laceration lesions of the spinal cord or whiplash shaken infant syndrome.

In addition, deprivation of oxygen or blood supply in general can cause acute injury as in hypoxia and/or ischemia including, but is not limited to, cerebrovascular insufficiency, cerebral ischemia or cerebral infarction (including cerebral ischemia or infarctions originating from embolic occlusion and thrombosis, retinal ischemia (diabetic or otherwise), glaucoma, retinal degeneration, multiple sclerosis, toxic and ischemic optic neuropathy, reperfusion following acute ischemia, perinatal hypoxic-ischemic injury, cardiac arrest or intracranial hemorrhage of any type (including, but not limited to, epidural, subdural, subarachnoid or intracerebral hemorrhage).

Trauma or injury to tissues of the nervous system may also take the form of more chronic and progressive neurodegenerative disorders, such as those associated with progressive neuronal cell death or compromise over a period of time including, but not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases (amyotrophic lateral sclerosis), multiple sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease or spinocerebellar ataxia type 3 and olivopontocerebellar degenerations, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome) or prion diseases (including, but not limited to Creutzfeld-Jakob disease, Gerstmann-Strussler-Scheinker disease, Kuru disease or fatal familial insomnia).

In addition, trauma and progressive injury to the nervous system can take place in various psychiatric disorders, including but not limited to, progressive, deteriorating forms of Bipolar disorder or Schizoaffective disorder or Schizophrenia, Impulse Control disorders, Obsessive Compulsive disorder (OCD), behavioral changes in Temporal Lobe Epilepsy and personality disorders.

In one preferred embodiment the compounds of the invention would be used to provide neuroprotection in disorders involving trauma and progressive injury to the nervous system in various psychiatric disorders. These disorders would be selected form the group consisting of; Schizoaffective disorder, Schizophrenia, Impulse Control disorders, Obsessive Compulsive disorder (OCD) and personality disorders.

In addition, trauma and injury make take the form of disorders associated with overt and extensive memory loss including, but not limited to, neurodegenerative disorders associated with age-related dementia, vascular dementia, diffuse white matter disease (Binswanger's disease), dementia of endocrine or metabolic origin, dementia of head trauma and diffuse brain damage, dementia pugilistica or frontal lobe dementia, including but not limited to Pick's Disease.

Other disorders associated with neuronal injury include, but are not limited to, disorders associated with chemical, toxic, infectious and radiation injury of the nervous system including the retina, injury during fetal development, prematurity at time of birth, anoxic-ischemia, injury from hepatic, glycemic, uremic, electrolyte and endocrine origin, injury of psychiatric origin (including, but not limited to, psychopathology, depression or anxiety), injury from peripheral diseases and plexopathies (including plexus palsies) or injury from neuropathy (including neuropathy selected from multifocal, sensory, motor, sensory-motor, autonomic, sensory-autonomic or demyelinating neuropathies (including, but not limited to Guillain-Barre syndrome or chronic inflammatory demyelinating polyradiculoneuropathy) or those neuropathies originating from infections, inflammation, immune disorders, drug abuse, pharmacological treatments, toxins, trauma (including, but not limited to compression, crush, laceration or segmentation traumas), metabolic disorders (including, but not limited to, endocrine or paraneoplastic), Charcot-Marie-Tooth disease (including, but not limited to, type 1a, 1b, 2, 4a or 1-X linked), Friedreich's ataxia, metachromatic leukodystrophy, Refsum's disease, adrenomyeloneuropathy, Ataxia-telangiectasia, Djerine-Sottas (including, but not limited to, types A or B), Lambert-Eaton syndrome or disorders of the cranial nerves).

Therefore, the term “neuroprotection” as used herein shall mean; inhibiting, preventing, ameliorating or reducing the severity of the dysfunction, degeneration or death of nerve cells, axons or their supporting cells in the central or peripheral nervous system of a mammal, including a human. This includes the treatment or prophylaxis of a neurodegenerative disease; protection against excitotoxicity or ameliorating the cytotoxic effect of a compound (for example, a excitatory amino acid such as glutamate; a toxin; or a prophylactic or therapeutic compound that exerts an immediate or delayed cytotoxic side effect including but not limited to the immediate or delayed induction of apoptosis) in a patient in need thereof.

Therefore, the term “a patient in need of treatment with a neuroprotective drug (NPD)” as used herein will refer to any patient who currently has or may develop any of the above syndromes or disorders, or any disorder in which the patient's present clinical condition or prognosis could benefit from providing neuroprotection to prevent the; development, extension, worsening or increased resistance to treatment of any neurological or psychiatric disorder.

The term “antiepileptic drug” (AED) will be used interchangeably with the term “anticonvulsant agent,” and as used herein, both terms refer to an agent capable of inhibiting (e.g., preventing slowing, halting, or reversing) seizure activity or ictogenesis when the agent is administered to a subject or patient.

The term “pharmacophore” is known in the art, and, as used herein, refers to a molecular moiety capable of exerting a selected biochemical effect, e.g., inhibition of an enzyme, binding to a receptor, chelation of an ion, and the like. A selected pharmacophore can have more than one biochemical effect, e.g., can be an inhibitor of one enzyme and an agonist of a second enzyme. A therapeutic agent can include one or more pharmacophore, which can have the same or different biochemical activities.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of the compounds or agents of the present invention to provide neuroprotection. In some instances, treatment with the compounds of the present invention will done in combination with other neuroprotective compounds or AED's to prevent, inhibit, or arrest the progression of neuronal death or damage or brain dysfunction or brain hyperexcitability.

The term “therapeutic effect” as used herein, refers to the effective provision of neuroprotection effects to prevent or minimize the death or damage or dysfunction of the cells of the patient's central or peripheral nervous system.

The term “a therapeutically effective amount” as used herein means a sufficient amount of one or more of the compounds of the invention to produce a therapeutic effect, as defined above, in a subject or patient in need of such neuroprotection treatment.

The terms “subject” or “patient” are used herein interchangeably and as used herein mean any mammal including but not limited to human beings including a human patient or subject to which the compositions of the invention can be administered. The term mammals include human patients and non-human primates, as well as experimental animals such as rabbits, rats, and mice, and other animals.

In some embodiments the methods of the present invention will be advantageously used to treat a patient who is not suffering or known to be suffering from a condition that is known in the art to be effectively treated with carbamate compounds or presently known neuroprotective compounds or AEDs. In these cases the decision to use the methods and compounds of the present invention would be made on the basis of determining if the patient is a “patient in need of treatment with a neuroprotective drug (NPD)”, as that term is defined above.

In some embodiments this invention provides methods of neuroprotection. In certain embodiments, these methods comprise administering a therapeutically effective amount of a carbamate compound of the invention to a patient who has not yet developed overt, clinical signs or symptoms of injury or damage to the cells of the nervous system but who may be in a high risk group for the development of neuronal damage because of injury or trauma to the nervous system or because of some known predisposition either biochemical or genetic or the finding of a verified biomarker of one or more of these disorders.

Thus, in some embodiments, the methods and compositions of the present invention are directed toward neuroprotection in a subject who is at risk of developing neuronal damage but who has not yet developed clinical evidence. This patient may simply be at “greater risk” as determined by the recognition of any factor in a subject's, or their families, medical history, physical exam or testing that is indicative of a greater than average risk for developing neuronal damage. Therefore, this determination that a patient may be at a “greater risk” by any available means can be used to determine whether the patient should be treated with the methods of the present invention.

Accordingly, in an exemplary embodiments, subjects who may benefit from treatment by the methods and compounds of this invention can be identified using accepted screening methods to determine risk factors for neuronal damage. These screening methods include, for example, conventional work-ups to determine risk factors including but not limited to:, for example, head trauma, either closed or penetrating, CNS infections, bacterial or viral, cerebrovascular disease including but not limited to stroke, brain tumors, brain edema, cysticercosis, porphyria, metabolic encephalopathy, drug withdrawal including but not limited to sedative-hypnotic or alcohol withdrawal, abnormal perinatal history including anoxia at birth or birth injury of any kind, cerebral palsy, learning disabilities, hyperactivity, history of febrile convulsions as a child, history of status epilepticus, family history of epilepsy or any a seizure related disorder, inflammatory disease of the brain including lupis, drug intoxication either direct or by placental transfer, including but not limited to cocaine poisoning, parental consanguinity, and treatment with medications that are toxic to the nervous system including psychotropic medications.

The determination of which patients may benefit from treatment with an NPD in patients who have no clinical signs or symptoms may be based on a variety of “surrogate markers” or “biomarkers”.

As used herein, the terms “surrogate marker” and “biomarker” are used interchangeably and refer to any anatomical, biochemical, structural, electrical, genetic or chemical indicator or marker that can be reliably correlated with the present existence or future development of neuronal damage. In some instances, brain-imaging techniques, such as computer tomography (CT), magnetic resonance imaging (MRI) or positron emission tomography (PET), can be used to determine whether a subject is at risk for neuronal damage.

Suitable biomarkers for the methods of this invention include, but are not limited to: the determination by MRI, CT or other imaging techniques, of sclerosis, atrophy or volume loss in the hippocampus or overt mesial temporal sclerosis (MTS) or similar relevant anatomical pathology; the detection in the patient's blood, serum or tissues of a molecular species such as a protein or other biochemical biomarker, e.g., elevated levels of ciliary neurotrophic factor (CNTF) or elevated serum levels of a neuronal degradation product; or other evidence from surrogate markers or biomarkers that the patient is in need of treatment with a neuroprotective drug.

It is expected that many more such biomarkers utilizing a wide variety of detection techniques will be developed in the future. It is intended that any such marker or indicator of the existence or possible future development of neuronal damage, as the latter term is used herein, may be used in the methods of this invention for determining the need for treatment with the compounds and methods of this invention.

A determination that a subject has, or may be at risk for developing, neuronal damage would also include, for example, a medical evaluation that includes a thorough history, a physical examination, and a series of relevant bloods tests. It can also include an electroencephalogram (EEG), CT, MRI or PET scan. A determination of an increased risk of developing neuronal damage or injury may also be made by means of genetic testing, including gene expression profiling or proteomic techniques. (See, Schmidt, D. Rogawski, M. A. Epilepsy Research 50; 71-78 (2002), and Loscher, W, Schmidt D. Epilepsy Research 50; 3-16 (2002))

For psychiatric disorders that may be stabilized or improved by a neuroprotective drug, e.g., Bipolar Disorder, Schizoaffective disorder, Schizophrenia, Impulse Control Disorders, etc. the above tests may also include a present state exam and a detailed history of the course of the patients symptoms such as mood disorder symptoms and psychotic symptoms over time and in relation to other treatments the patient may have received over time, e.g., a life chart. These and other specialized and routine methods allow the clinician to select patients in need of therapy using the methods and formulations of this invention.

In some embodiments of the present invention carbamate compounds suitable for use in the practice of this invention will be administered either singly or concomitantly with at least one or more other compounds or therapeutic agents, e.g., with other neuroprotective drugs or antiepileptic drugs, anticonvulsant drugs. In these embodiments, the present invention provides methods to treat or prevent neuronal injury in a patient. The method includes the step of; administering to a patient in need of treatment, an effective amount of one of the carbamate compounds disclosed herein in combination with an effective amount of one or more other compounds or therapeutic agents that have the ability to provide neuroprotection or to treat or prevent seizures or epileptogenesis or the ability to augment the neuroprotective effects of the compounds of the invention.

“Concomitant administration” or “combination administration” of a compound, therapeutic agent or known drug with a compound of the present invention means administration of the drug and the one or more compounds at such time that both the known drug and the compound will have a therapeutic effect. In some cases this therapeutic effect will be synergistic. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

The said one or more other compounds or therapeutic agents may be selected from compounds that have one or more of the following properties: antioxidant activity; NMDA receptor antagonist activity, augmentation of endogenous GABA inhibition; NO synthase inhibitor activity; iron binding ability, e.g., an iron chelator; calcium binding ability, e.g., a Ca (II) chelator; zinc binding ability, e.g., a Zn (II) chelator; the ability to effectively block sodium or calcium ion channels, or to open potassium or chloride ion channels in the CNS of a patient.

In some preferred embodiments, the one or more other compounds or therapeutic agents would antagonize NMDA receptors by binding to the NMDA receptors (e.g., by binding to the glycine binding site of the NMDA receptors) and/or the agent would augment GABA inhibition by decreasing glial GABA uptake.

In addition the said one or more other compounds or therapeutic agents may be any agent known to suppress seizure activity even if that compound is not known to provide neuroprotection. Such agents would include but not be limited to any effective AED known to one of skill in the art or discovered in the future, for example suitable agents include, but are not limited to; carbamazepine, clobazam, clonazepam, ethosuximide, felbamate, gabapentin, lamotigine, levetiracetam, oxcarbazepine, phenobarbital, phenytoin, pregabalin, primidone, retigabine, talampanel, tiagabine, topiramate, valproate, vigabatrin, zonisamide, benzodiazepines, barbiturates and sedative hypnotics in general.

In addition, in some embodiments, the compounds of this invention will be used, either alone or in combination with each other or in combination with one or more other therapeutic medications as described above, or their salts or esters, for manufacturing a medicament for the purpose of providing neuroprotection to a patient or subject in need thereof.

Carbamate Compounds as Pharmaceuticals:

The present invention provides enantiomeric mixtures and isolated enantiomers of Formula 1 and/or Formula 2 as pharmaceuticals. The carbamate compounds are formulated as pharmaceuticals to provide neuroprotection in a subject.

In general, the carbamate compounds of the present invention can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection.) Administration of the compounds directly to the nervous system can include, for example, administration to intracerebral, intraventricular, intacerebroventricular, intrathecal, intracisternal, intraspinal or peri-spinal routes of administration by delivery via intracranial or intravertebral needles or catheters with or without pump devices.

In addition, in the case of diseases or disorders of the eye including but not limited to; retinal ischemia (diabetic or otherwise), glaucoma, retinal degeneration, macular degeneration, multiple sclerosis, toxic and ischemic optic neuropathy the compounds of the present invention, including combinations of compounds, can be administered by means of direct exogenous application to the eye, i.e., to the sclera or otherwise, e.g., eye drops or by ocular implant or other slow delivery device including microspheres including by direct injection into the vitreous humor etc.

Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso AR: Reminqton's Pharmaceutical Sciences, 17th ed., Mack Publishing Company, Easton Pa., 1985, the disclosure of which is incorporated herein by reference in its entirety and for all purposes. Suitable liquid carriers, especially for injectable solutions, include water, aqueous saline solution, aqueous dextrose solution, and glycols.

The carbamate compounds can be provided as aqueous suspensions. Aqueous suspensions of the invention can contain a carbamate compound in admixture with excipients suitable for the manufacture of aqueous suspensions. Such excipients can include, for example, a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate).

The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions for use in the present methods can be formulated by suspending a carbamate compound in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these.

Suitable emulsifying agents include naturally occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations of the present invention suitable for parenteral administration, such as, for example, by intraarticular (in the joints), intravenous, intramuscular, intradermal, intraperitoneal, and subcutaneous routes, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter.

Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The concentration of a carbamate compound in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally acceptable diluents or solvent, such as a solution of 1,3-butanediol. The formulations of commends can be presented in unit-dose or multi-dose sealed containers, such as ampoules and vials. Injection solutions and suspensions can be prepared from sterile powders, granules, and tablets of the kind previously described.

A carbamate compound suitable for use in the practice of this invention can be and is preferably administered orally. The amount of a compound of the present invention in the composition can vary widely depending on the type of composition, size of a unit dosage, kind of excipients, and other factors well known to those of ordinary skill in the art. In general, the final composition can comprise, for example, from 0.000001 percent by weight (% w) to 10% w of the carbamate compound, preferably 0.00001% w to 1% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient.

Formulations suitable for oral administration can consist of (a) liquid solutions, such as an effective amount of the pharmaceutical formulation suspended in a diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen. If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compounds of the present invention can also be administered in the form of suppositories for rectal administration of the drug. These formulations can be prepared by mixing the drug with a suitable non-irritating excipient that is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The compounds of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention.

In another embodiment, the compounds of the present invention can be delivered by the use of liposomes which fuse with the cellular membrane or are endocytosed, i.e., by employing ligands attached to the liposome that bind to surface membrane protein receptors of the cell resulting in endocytosis. By using liposomes, particularly where the liposome surface carries ligands specific for target cells, or are otherwise preferentially directed to a specific organ, one can focus the delivery of the carbamate compound into target cells in vivo. (See, e.g., Al-Muhammed, J. Microencapsul. 13:293-306, 1996; Chonn, Curr. Opin. Biotechnol. 6:698-708, 1995; Ostro, Am. J Hosp. Pharm. 46:1576-1587, 1989).

The pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation can be a lyophilized powder which can contain, for example, any or all of the following: 1 mM-50 mM histidine, 0.1%-2% sucrose, 2%-7% mannitol, at a pH range of 4.5 to 5.5, that is combined with buffer prior to use.

Pharmaceutically acceptable salts and esters refers to salts and esters that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like. Pharmaceutically acceptable salts can also include acid addition salts formed from the reaction of amine moieties in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds. When there are two acidic groups present, a pharmaceutically acceptable salt or ester may be a mono-acid-mono-salt or ester or a di-salt or ester; and similarly where there are more than two acidic groups present, some or all of such groups can be salified or esterified.

Compounds named in this invention can be present in unsalified or unesterified form, or in salified and/or esterified form, and the naming of such compounds is intended to include both the original (unsalified and unesterified) compound and its pharmaceutically acceptable salts and esters. The present invention includes pharmaceutically acceptable salt and ester forms of Formula 1 and Formula 2. More than one crystal form of an enantiomer of Formula 1 or Formula 2 can exist and as such are also included in the present invention.

A pharmaceutical composition of the invention can optionally contain, in addition to a carbamate compound, at least one other therapeutic agent useful in the treatment of a disease or condition associated with providing neuroprotection.

Methods of formulating pharmaceutical compositions have been described in numerous publications such as Pharmaceutical Dosage Forms: Tablets. Second Edition. Revised and Expanded. Volumes 1-3, edited by Lieberman et al; Pharmaceutical Dosaqe Forms: Parenteral Medications. Volumes 1-2, edited by Avis et al; and Pharmaceutical Dosage Forms: Disperse Systems. Volumes 1-2, edited by Lieberman et al; published by Marcel Dekker, Inc, the disclosure of which are herein incorporated by reference in their entireties and for all purposes.

The pharmaceutical compositions are generally formulated as sterile, substantially isotonic and in full compliance with all Good Manufacturing Practice (GMP) regulations of the U.S. Food and Drug Administration.

Dosage Regimens

The present invention provides methods of providing neuroprotection in a mammal using carbamate compounds. The amount of the carbamate compound necessary to provide neuroprotection is defined as a therapeutically or a pharmaceutically effective dose. The dosage schedule and amounts effective for this use, i.e., the dosing or dosage regimen will depend on a variety of factors including the stage of the disease, the patient's physical status, age and the like. In calculating the dosage regimen for a patient, the mode of administration is also taken into account.

A person of ordinary skill in the art will be able without undue experimentation, having regard to that skill and this disclosure, to determine a therapeutically effective amount of a particular substituted carbamate compound for practice of this invention (see, e.g., Lieberman, Pharmaceutical Dosage Forms (Vols. 1-3, 1992); Lloyd, 1999, The art, Science and Technology of Pharmaceutical Compounding; and Pickar, 1999, Dosage Calculations). A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds.

For treatment purposes, the compositions or compounds disclosed herein can be administered to the subject in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times daily, 3 times per week, or weekly. In one embodiment of the present invention, the pharmaceutical formulations of the present invention are orally administered once or twice daily.

A treatment regimen with the compounds of the present invention can commence, for example, after a subject suffers from a brain damaging injury or other initial insult but before the subject is diagnosed with epilepsy or other manifestation of neuronal injury. In one embodiment, a subject that is identified as being at a high risk of developing neuronal injury or a subject having a disease associated with a risk of developing neuronal damage, e.g., neonatal hypoxia, can commence a treatment regimen with a carbamate compound of the present invention.

In certain embodiments, the carbamate compound can be administered daily for a set period of time (week, month, year) after occurrence of the brain damaging injury or initial insult. An attendant physician will know how to determine that the carbamate compound has reached a therapeutically effective level, e.g., clinical exam of a patient, or by measuring drug levels in the blood or cerebro-spinal fluid.

In this context, a therapeutically effective dosage of the biologically active agent(s) can include repeated doses within a prolonged treatment regimen that will yield clinically significant results to provide neuroprotection. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

In an exemplary embodiment of the present invention, unit dosage forms of the compounds are prepared for standard administration regimens. In this way, the composition can be subdivided readily into smaller doses at the physician's direction. For example, unit dosages can be made up in packeted powders, vials or ampoules and preferably in capsule or tablet form.

The active compound present in these unit dosage forms of the composition can be present in an amount of, for example, from about 10 mg. to about one gram or more, for single or multiple daily administration, according to the particular need of the patient. By initiating the treatment regimen with a minimal daily dose of about one gram, the blood levels of the carbamate compounds can be used to determine whether a larger or smaller dose is indicated.

Effective administration of the carbamate compounds of this invention can be administered, for example, at an oral or parenteral dose of from about 0.01 mg/kg/dose to about 150 mg/kg/dose. Preferably, administration will be from about 0.1/mg/kg/dose to about 25 mg/kg/dose, more preferably from about 0.2 to about 18 mg/kg/dose. Therefore, the therapeutically effective amount of the active ingredient contained per dosage unit as described herein can be, for example, from about 1 mg/day to about 7000 mg/day for a subject having, for example, an average weight of 70 kg.

The methods of this invention also provide for kits for use in providing neuroprotection. After a pharmaceutical composition comprising one or more carbamate compounds of this invention, with the possible addition of one or more other compounds of therapeutic benefit, has been formulated in a suitable carrier, it can be placed in an appropriate container and labeled for providing neuroprotection. Additionally, another pharmaceutical comprising at least one other therapeutic agent useful in the provide neuroprotection, treatment of epileptogenesis, epilepsy or another disorder or condition associated with neuronal injury can be placed in the container as well and labeled for treatment of the indicated disease. Such labeling can include, for example, instructions concerning the amount, frequency and method of administration of each pharmaceutical.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. The following examples are provided to illustrate specific aspects of the invention and are not meant to be limitations.

### EXAMPLES

The activities of a compound of Formula (I) and Formula (II) for use in providing neuroprotection were evaluated in the following experimental examples. In examples 1 and 2 the activity of an isolated S-enantiomer of Formula 1 (e.g., Formula 7), herein referred to as the “test compound” (TC), was evaluated in a rat model of induced epilepsy to determine the efficacy of the compound for neuroprotection and in the treatment of epileptogenesis in the model of temporal lobe epilepsy induced by lithium and pilocarpine in the rat. These examples are intended to be a way of illustrating various embodiments of the invention but not intended to limit the invention in any way.

### Example 1

The Lithium-pilocarpine Model of Temporal Lobe Epilepsy

The model induced in rats by pilocarpine associated with lithium (Li-Pilo) reproduces most of the clinical and neurophysiological features of human temporal lobe epilepsy (Turski et al., 1989, Synapse 3:154-171; Cavalheiro, 1995, Ital J Neurol Sci 16:33-37). In adult rats, the systemic administration of pilocarpine leads to status epilepticus (SE). The lethality rate reaches 30-50% during the first days. In the surviving animals, neuronal damage predominates within the hippocampal formation, the piriform and entorhinal cortices, thalamus, amygdaloid complex, neocortex and substantia nigra. This acute seizure period is followed by a “silent” seizure-free phase lasting for a mean duration of 14-25 days after which all animals exhibit spontaneous recurrent convulsive seizures at the usual frequency of 2 to 5 per week (Turski et al., 1989, Synapse 3:154-171; Cavalheiro, 1995, Ital J Neurol Sci 16:33-37; Dube et al., 2001, Exp Neurol 167:227-241).

Lithium-pilocarpine and Treatments with the Test Compound

Male Wistar rats weighing 225-250 g, provided by Janvier Breeding Center (Le Genest-St-lste, France) were housed under controlled standard conditions (light/dark cycle, 7.00 a.m.-7.00 p.m. lights on), with food and water available ad libitum. All animal experimentation was performed in accordance with the rules of the European Communities Council Directive of Nov. 24,1986 (86/609/EEC), and the French Department of Agriculture (License N° 67-97). For electrode implantation, rats were anesthetized by an i.p. injection of 2.5 mg/kg diazepam (DZP, Valium, Roche, France) and 1 mg/kg ketamine chlorhydrate (Imalgene 1000, Rhone Merrieux, France). Four single-contact recording electrodes were placed on the skull, over the parietal cortex, two on each side.

Status Epilepticus Induction:

Treatment with the Test Compound and Occurrence of Spontaneous Recurrent Seizures (SRS)

All rats received lithium chloride (3 meq/kg, i.p., Sigma, St Louis, Mo., U.S.A.); about 20 h later, animals were placed into plexiglas boxes, in order to record baseline cortical EEG. Methylscopolamine bromide (1 mg/kg, s.c., Sigma) was administered to limit the peripheral effects of the convulsant. SE was induced by injecting pilocarpine hydrochloride (25 mg/kg, s.c., Sigma) 30 min after methyl-scopolamine. The bilateral EEG cortical activity was recorded during the whole duration of SE and behavioral changes were noted.

The effects of increasing doses of the test compound were studied on 3 groups of rats. The animals of the first group received 10 mg/kg of the test compound, i.p., 1 h after the onset of SE (pilo-TC10) while the animals of groups 2 and 3 received 30 and 60 mg/kg of the test compound (pilo-TC30 and pilo-TC60), respectively.

Another group was injected with 2 mg/kg diazepam (DZP, i.m.) at 1 h after the onset of SE. This is a standard treatment to improve animals survival after SE (pilo-DZP). The control group received saline instead of pilocarpine and the test compound (saline-saline). The pilo-test compound rats surviving SE were then injected about 10 h after the first test compound injection with a second i.p. injection of the same dose of the test compound and were maintained under a twice daily treatment with the test compound for 6 additional days. Pilo-DZP received a second injection of 1 mg/kg DZP on the day of SE at about 10 h after the first one. Thereafter, Pilo-DZP and saline-saline rats received twice daily an equivalent volume of saline.

The effects of the test compound on the EEG and on the latency to occurrence of SRS were investigated by daily video recording of the animals for 10 h per day and the recording of the electrographic activity twice a week for 8 h.

Quantification of Cell Densities

Quantification of cell densities was performed at 6 days after SE on 8 pilo-DZP, 8 pilo-TC10, 7 pilo-TC30, 7 pilo-TC60, and 6 saline-saline rats. At 14 days after SE, animals were deeply anesthetized with 1.8 g/kg pentobarbital (Dolethal®, Vetoquinol, Lure, France. Brains were then removed and frozen. Serial 20 μm slices were cut in a cryostat, air-dried during several days before thionine staining.

Quantification of cell densities was performed with a 10×10 boxes 1 cm² microscopic grid on coronal sections according to the stereotaxic coordinates of the rat brain atlas (Paxinos and Watson, 1986, The Rat brain in Stereotaxic Coordinates, 2^{nd} ed. Academic Press, San Diego). Cell counts were performed twice in a blind manner and were the average of at least 3 values from 2 adjacent sections in each individual animal. Counts involved only cells larger than 10 μm, smaller ones being considered as glial cells.

Timm Staining

At 2 months after the onset of spontaneous recurrent seizures, mossy fiber sprouting was examined on rats in the chronic period exposed to the test compound or DZP and in 3 saline-saline rats. Animals were deeply anaesthetized and perfused transcardially with saline followed by 100 ml of 1.15% (w/v) Na₂S in 0.1 M phosphate buffer, and 100 ml of 4% (v/v) formaldehyde in 0.1 M phosphate buffer. Brains were removed from skull, post-fixed in 4% formaldehyde during 3-5 h and 40 μm sections were cut on a sliding vibratome and mounted on gelatin-coated slides.

The following day, sections were developed in the dark in a 26° C. solution of 50% (w/v) arabic gum (160 ml), sodium citrate buffer (30 ml), 5.7% (w/v) hydroquinone (80 ml) and 10% (w/v) silver nitrate (2.5 ml) during 40-45 min. The sections were then rinsed with tap water at 40° C. during at least 45 min, rinsed rapidly with distilled water and allowed to dry. They were dehydrated in ethanol and coverslipped.

Mossy fiber sprouting was evaluated according to criteria previously described in dorsal hippocampus (Cavazos et al., 1991, J Neurosci 11:2795-2803.), which are follows: 0—no granules between the tips and crest of the DG; 1—sparse granules in the supragranular region in a patchy distribution between the tips and crest of DG; 2—more numerous granules in a continuous distribution between the tips and crest of DG; 3—prominent granules in a continuous pattern between tips and crest, with occasional patches of confluent granules between tips and crest; 4—prominent granules that form a confluent dense laminar band between tips and crest and 5—confluent dense laminar band of granules that extends into the inner molecular layer.

Data Analysis

For the comparison of the characteristics of SE in pilo-saline and pilo-test compound animals, a non-paired Student's t-test was used. The comparison between the number of rats seizing in both groups was performed by means of a Chi square test. For neuronal damage, statistical analysis between groups was performed using ANOVA followed by a Fisher's test for multiple comparisons using the Statview software (Fisher R A, 1946a, Statistical Methods for Research Workers (10th edition) Oliver & Boyd, Edinburgh; Fisher R A, 1946b, The Design of Experiments (4th edition) Oliver & Boyd, Edinburgh)

Behavioral and EEG Characteristics of Lithium-pitocarnine Status Epilepticus

A total number of Sprague-Dawley rats weighing 250-330 g were subjected to Li-pilo induced SE. The behavioral characteristics of SE were identical in both pilo-saline and pilo-test compound groups. Within 5 min after pilocarpine injection, rats developed diarrhea, piloerection and other signs of cholinergic stimulation. During the following 15-20 min, rats exhibited head bobbing, scratching, chewing and exploratory behavior. Recurrent seizures started around 15-20 min after pilocarpine administration. These seizures which associated episodes of head and bilateral forelimb myoclonus with rearing and falling progressed to SE at about 35-40 min after pilocarpine, as previously described (Turski et al., 1983, Behav Brain Res 9:315-335.).

EEG Patterns During SE

During the first hour of SE, in the absence of pharmacological treatment, the amplitude of the EEG progressively increased while the frequency decreased. Within 5 min after the injection of pilocarpine, the normal background EEG activity was replaced with low voltage fast activity in the cortex while theta rhythm (5-7 Hz) appeared in the hippocampus. By 15-20 min, high voltage fast activity superposed over the hippocampal theta rhythm and isolated high voltage spikes were recorded only in the hippocampus while the activity of the cortex did not substantially change.

By 35-40 min after pilocarpine injection, animals developed typical electrographic seizures with high voltage fast activity present in both the hippocampus and cortex which first occurred as bursts of activity preceding seizures and were followed by continuous trains of high voltage spikes and polyspikes lasting until the administration of DZP or the test compound. At about 3-4 h of SE, the hippocampal EEG was characterized by periodic electrographic discharges (PEDs, about one/sec) in the pilo-DZP and in the pilo-10 group in both hippocampus and cortex. The amplitude of EEG background activity was low in the pilo-TC60 animals. By 6-7 h of SE, spiking activity was still present in the cortex and the hippocampus in the DZP-and TC10-treated rats while the amplitude of the EEG decreased and came back to baseline levels in the hippocampus of TC30 rats and in both structures of TC60 treated rats. There was no difference between TC10, TC30, and TC60 groups. By 9 h of SE, isolated spikes were still recorded in the hippocampus of test compound-treated rats and occasionally in the cortex. In both structures, the background activity was of very low amplitude at that time.

Mortality Induced by SE

During the first 48 h after SE, the degree of mortality was similar in pilo-DZP rats (23%, 5/22), pilo-TC10 rats (26%, 6/23), and pilo-TC30 rats (20%, 5/25), The mortality rate was largely reduced in pilo-TC60 rats in which it only reached 4% (1/23). The difference was statistically significant (p<0.01).

EEG Characteristics of the Silent Phase and Occurrence of Spontaneous Recurrent Seizures

The EEG patterns during the silent period were similar in pilo-DZP and pilo-TC10, 30 or 60 rats. At 24 and 48 h days after SE, the baseline EEG was still characterized by the occurrence of PEDs on which large waves or spikes could be superimposed. Between 1 and 8 h after injection of the test compound or vehicle injection, there was no change in the pilo-DZP or pilo-TC10 groups. In TC30 and TC60 rats, the frequency and amplitude of PEDs decreased as soon as 10 min after injection and were replaced by spikes of large amplitude in the TC30 group and of low amplitude in the TC60 group. By 4 h after injection the EEG had returned to baseline levels in the two latter groups. By 6 days after SE, the EEG was still of lower amplitude than before pilocarpine injection and in most groups spikes could still be recorded, occasionally in the pilo-DZP, -TC10 and -TC30 rats. In pilo-TC60 rats, the frequency of large amplitude spikes was higher than in all other groups. After the TC compound or vehicle-injection, EEG recording was not affected by the injection in the pilo-DZP and pilo-TC10 groups. In pilo-TC30 rats, the injection induced the occurred of slow waves on the EEG of both hippocampus and cortex and a decreased frequency of spikes in the pilo-TC60 rats.

All the rats exposed to DZP, TC10 and TC30 and studied until the chronic phase developed SRS with a similar latency. The latency was 18.2±6.9 days (n=9) in pilo-DZP rats, 15.4±5.1 days (n=7) in pilo-TC10 rats, 18.9±9.0 days (n=10) in pilo-TC30 rats. In the group of rats subjected to TC60, a subgroup of rats became epileptic with a latency similar to that of the other groups, i.e. 17.6±8.7 days (n=7) while another group of rats became epileptic with a much longer delay ranging from 109 to 191 days post-SE (149.8±36.0 days, n=4) and one rat did not become epileptic in a delay of 9 months post-SE. The difference in the latency to SRS between pilo-DZP, pilo-TC10, pilo-TC30 and the first subgroup of pilo-TPM60 rats was not statistically significant. None of the saline-saline rats (n=5) developed SRS.

To calculate the frequency of SRS in pilocarpine-exposed rats, the seizure severity and distinguished stage III (clonic seizures of facial muscles and anterior limbs) and stage IV-V seizures (rearing and falling) was considered. The frequency of stage III SRS per week in pilo-DZP and pilo-test compound rats was variable amongst the groups. It was low, constant in the pilo-DZP and pilo-TC60 (with early SRS onset) groups during the first 3 weeks and had disappeared during the 4th week in the pilo-DZP group. The frequency of stage III SRS was higher in the pilo-TC10 group where it was significantly increased over pilo-DZP values during weeks 3 and 4. The frequency of more severe stage IV-V SRS was highest during the first week in most groups, except pilo-TC30 and TC60 with late seizure onset where the SRS frequency was constant over the whole 4 weeks in TC30 group and over the first two weeks in the pilo-TC60 group with late SRS onset in which no stage IV-V seizures where no seizures recorded after the second week. The frequency of stage IV-V SRS was significantly reduced in the TC10, TC30 and TC60 (with early SRS onset) groups (2.3-6,1 SRS per week) compared to the pilo-DZP group (11.3 SRS per week) during the first week. During weeks 2-4v the frequency of stage IV-V SRS was reduced in all groups compared to the first week reaching values of 2-6 seizures per week, except in the pilo-TC60 group with early SRS onset where the frequency of seizures was significantly reduced to 0.6-0.9 seizure per week compared to the pilo-DZP group in which the frequency of SRS ranged from 3.3 to 5.8.

Cell Densities in Hippocampus, Thalamus and Cortex

In pilo-DZP rats compared to saline-saline rats, the number of cells was massively decreased in the CA1 region of the hippocampus (70% cell loss in the pyramidal cell layer) while the CAS region was less extensively damaged (54% cell loss in CA3a and 31% in CA3b). In the dentate gyrus, the pilo-DZP rats experienced extensive cell loss in the hilus (73%) while the granule cell layer did not show visible damage. Similar damage was observed in the ventral hippocampus but cell counts were not performed in this region. Extensive damage was also recorded in the lateral thalamic nucleus (91% cell loss) while the mediodorsal thalamic nucleus was more moderately damaged (56%). In the piriform cortex, cell loss was total in layers III-IV which was no longer visible and reached 53% in layer II in pilo-DZP rats. In the dorsal entorhinal cortex, layers II and III-IV underwent slight damage (9 and 15%, respectively). Layer II of the ventral entorhinal cortex was totally preserved while layers III-IV underwent a 44% cell loss.

In the hippocampus of pilo-test compound animals, cell loss was reduced compared to pilo-DZP rats in the CA1 pyramidal layer in which the cell loss reached 75% in pilo-DZP and 35 and 16% in the pilo-TC30 or pilo-TC60 animals, respectively. This difference was statistically significant at the two test compound doses. In the CAS pyramidal layer, the test compound did not afford any protection in the CA3a area while the 60 mg/kg of the test compound dose was significantly neuroprotective in CA3b. In the dentate gyrus, the cell loss in the hilus was similar in pilo-test compound (69-72%) and pilo-DZP animals (73%). In the two thalamic nuclei, the 60 mg/kg dose was also protective in reducing neuronal damage by 65 and 42% in the lateral and mediodorsal nucleus, respectively. In the cerebral cortex, the treatment with the test compound afforded neuronal protection compared to DZP only at the highest dose, 60 mg/kg. At the two lowest doses, 10 and 30 mg/kg, the total loss of cells and tissue disorganization observed in layers III-IV of the piriform cortex was identical in pilo-DZP rats and pilo-test compound rats and did not allow any counting in any of the groups. In layers II and III-IV of the piriform cortex, the TC60 treatment reduced neuronal damage recorded in the pilo-DZP rats by 41 and 44%, respectively. In the ventral entorhinal cortex, neuroprotection was induced by TC60 administration in layers III-IV and reached 31% compared to pilo-DZP rats. In the entorhinal cortex, there was a slight worsening of cell loss in pilo-TC10 rats compared with pilo-DZP rats in layers III-IV of the dorsal entorhinal cortex (28% more damage) and layers III-IV of the ventral entohinal cortex (35% more damage). At the other doses of the test compound, cell loss in the entorhinal cortex was similar to the one recorded in pilo-DZP rats.

Mossy Fiber Sprouting in Hippocampus

All rats exhibiting SRS in pilo-DZP and pilo-TPM groups showed similar intensity of Timm staining in the inner molecular layer of the dentate gyrus (scores 2-4). Timm staining was present both on the upper and lower blades of the dentate gyrus. The mean value of the Timm score in the upper blade reached 2.8±0.8 in pilo-DZP rats (n=9), 1.5±0.6 in pilo-TC10 rats (n=7), 2.6±1.0 in pilo-TC30 rats (n=10), and 1.5±0.7 in the whole group of pilo-TC60 rats (n=11). When the pilo-TC60 group was subdivided according to the latency to SRS, the subgroup with early SRS occurrence showed a Timm score of 1.8±0.6 (n=6) and the subgroup of rats with late occurrence or absence of SRS had a Timm score of 1.2±0.6 (n=5). The values recorded in the pilo-DZP rats were statistically significantly different from the values in the pilo-TC10 (p=0.032) and the pilo-TC60 subgroup with late or no seizures (p=0.016).

Discussion and Conclusions

The results of the present study show that a 7-day treatment with the test compound starting at 1 h after the onset of SE is able to protect some brain areas from neuronal damage, e.g., in the pyramidal cell layer of the CA1 and CA3b area, the mediodorsal thalamus, layers II and I1MV of the piriform cortex and layers III-IV of the ventral entorhinal cortex, but only at the highest dose the test compound, i.e. 60 mg/kg. The latter dose of the test compound is also able to delay the occurrence of SRS, at least in a subgroup of animals that became epileptic with a mean delay that was about 9-fold longer than in the other groups of animals and one animal did not become epileptic in a delay of 9 months after SE.

These results show that one compound with anti-ictal properties, which are the classical properties of most antiepileptic-marketed drugs, is also able to delay epileptogenesis, i.e. to be antiepileptogenic. The data of the present study show also that the test compound treatment, whatever the dose used, decreases the severity of the epilepsy since it decreases the number of stage IV-V seizures, mainly during the first week of occurrence and during the whole period of 4-weeks observation with the TC60 treatment. Moreover, in the TC10 group, there is a shift to an increase in the occurrence of less severe stage III seizures that are more numerous than in the pilo-DZP group.

### Example 2

The aim of the present project was to pursue the study of the potential neuroprotective and antiepileptogenic properties of the test compound (TC) in the lithium-pilocarpine (Li-Pilo) model of temporal lobe epilepsy. This study follows a first one described in Example 1 in which it was shown that TC was able to protect areas CA1 and CA3 of the hippocampus, piriform and ventral entorhinal cortex from neuronal damage induced by Li-Pilo status epilepticus (SE). Most of these neuroprotective properties occurred at the highest dose studied, 60 mg/kg and the treatment was able to delay the occurrence of spontaneous seizures in 36% (4 out of 11) of the rats. In the present study, we propose to study the consequences of treatment by higher doses of TC on neuronal damage and epileptogenesis.

The Lithium-pilocarpine Model of Temporal Lobe Epilepsy

The model of epilepsy induced in rats by pilocarpine associated with lithium (Li-Pilo) reproduces most of the clinical and neurophysiological features of human temporal lobe epilepsy (Turski et al., 1989; Cavalheiro, 1995). In adult rats, the systemic administration of pilocarpine leads to SE which may last for up to 24 h. The lethality rate reaches 30-50% during the first days. In the surviving animals, neuronal damage predominates within the hippocampal formation, the piriform and entorhinal cortices, thalamus, amygdaloid complex, neocortex and substantia nigra. This acute seizure period is followed by a “silent” seizure-free phase lasting for a mean duration of 14-25 days after which all animals exhibit spontaneous recurrent convulsive seizures at the usual frequency of 2 to 5 per week (Turski et al., 1989; Cavalheiro, 1995; Dubé et al., 2001). The current antiepileptic drugs do not prevent the epileptogenesis and are only transiently efficient on recurrent seizures.

In our previous study, we studied the potential neuroprotective and antiepileptogenic effects of increasing doses of TC given in monotherapy and compared to our standard diazepam (DZP) treatment mostly given to prevent high mortality. These data show that a 7-day treatment with 10, 30 or 60 mg/kg TC starting at 1 h after the onset of SE is able to protect some brain areas from neuronal damage. This effect is statistically significant in the pyramidal cell layer of the CA1 and CA3b area, the mediodorsal thalamus, layers II and III-IV of the piriform cortex and layers III-IV of the ventral entorhinal cortex, but only at the highest dose of TC, i.e. 60 mg/kg. Moreover, it appears that the latter dose of TC is also the only one that is able to delay the occurrence of SRS, at least in a subgroup of animals that became epileptic with a mean delay that was about 9-fold longer than in the other groups of animals and one animal did not become epileptic in a delay of 9 months after SE.

In the present study, the effects of different doses of TC, i.e. 30, 60, 90 and 120 mg/kg using the same design as in the previous study were tested. The treatment was started one hour after the onset of SE and the animals were treated with a second injection of the same dose of the drug. This early treatment of SE was followed by a 6 days TC treatment. This report concerns the effects of the four different doses of TC on neuronal damage assessed in hippocampus, parahippocampal cortices, thalamus and amygdala at 14 days after SE and on the latency to and frequency of spontaneous epileptic seizures.

Animals

Adult male Sprague-Dawley rats provided by Janvier Breeding Center (Le Genest-St-Isle, France) were housed under controlled, uncrowded standard conditions at 20-22° C. (light/dark cycle, 7.00 a.m.-7.00 p.m. lights on), with food and water available ad libitum. All animal experimentation was performed in accordance with the rules of the European Communities Council Directive of Nov. 24, 1986 (86/609/EEC), and the French Department of Agriculture (License N° 67-97).

Status Epilepticus Induction, TC Treatment and Occurrence of SRS

All rats received lithium chloride (3 meq/kg, i.p., Sigma, St Louis, Mo., U.S.A.) and about 20 h later, all animals received also methylscopolamine bromide (1 mg/kg, s.c., Sigma) that was administered to limit the peripheral effects of the convulsant. SE was induced by injecting pilocarpine hydrochloride (25 mg/kg, s.c., Sigma) 30 min after methylscopolamine. The effects of increasing doses of TC (RWJ) were studied in 5 groups of rats. The animals received either 2.5 mg/kg DZP, i.m., or 30, 60, 90 or 120 mg/kg TC (TC30, TC60, TC90, TC120 0), i.p., at 1 h after the onset of SE. The control group received vehicle instead of pilocarpine and TC. The rats surviving SE were then injected about 10 h after the first TC injection with a second i.p. injection of 1.25 mg/kg DZP for the DZP group or of the same dose of TC as in the morning and were maintained under a twice daily TC treatment (s.c.) for 6 additional days while DZP rats received a vehicle injection.

The effects of DZP and the 4 doses of TC on epileptogenesis were investigated by daily video recording of the animals for 10 h per day. Video recording was performed for 4 weeks during which the occurrence of the first seizure was noted as well as the total number of seizures over the whole period. Animals were then taken off the video recording system and kept for 4 additional weeks in our animal facilities before they were sacrificed after a total period of 8 weeks of epilepsy. The rats that did not exhibited seizures were sacrificed after 5 months of video recording.

Quantification of Cell Densities

Quantification of cell densities was performed at two times after SE: a first group was studied 14 days after SE and was composed by 7 DZP, 8 TC30, 11 TC60, 10 TC90, 8 TC120 and 8 control rats not subjected to SE. A second group used for the study of the latency to SRS was sacrificed either 8 weeks after the first SRS or at 5 months when no SRS could be seen in that delay and was composed by 14 DZP, 8 TC30, 10 TC60, 11 TC90, 9 TC120 rats. At the moment, neuronal counting is still in progress in the second group of animals studied for epileptogenesis and long-term counting and the data concerning that part of the study will not be included in the present report. For neuronal counting, animals were deeply anesthetized with 1.8 g/kg pentobarbital (Dolethal®, Vétoquinol, Lure, France). Brains were then removed and frozen. Serial 20 μm slices were cut in a cryostat, air-dried during several days before thionine staining. Quantification of cell densities was performed with a 10×10 boxes 1 cm² microscopic grid on coronal sections according to the stereotaxic coordinates of the rat brain atlas (Paxinos and Watson, 1986). The grid of counting was placed on a well defined area of the cerebral structure of interest and counting was carried out with a microscopic enlargement of 200- or 400-fold defined for each single cerebral structure. Cell counts were performed twice on each side of three adjacent sections for each region by a single observer unaware of the animal's treatment. The number of cells obtained in the 12 counted fields in each cerebral structure was averaged. This procedure was used to minimize the potential errors that could result from double counting leading to overestimation of cell numbers. Neurons touching the inferior and right edges of the grid were not counted. Counts involved only neurons with cell bodies larger than 10 μm. Cells with small cell bodies were considered as glial cells and were not counted.

Data Analysis

For neuronal damage and epileptogenesis, statistical analysis between groups was performed by means of a one-way analysis of variance followed by a post-hoc Dunnett or Fisher test using the Statistica software.

Results

Behavioral Characteristics of Lithium-pilocarpine Status Epilepticus

A total number of 143 Sprague-Dawley rats weighing 250-330 g were subjected to lithium-pilocarpine (Li-pilo)-induced SE. In this number 10 did not develop SE while 133 rats developed a full characteristic Li-pilo SE. The behavioral characteristics of SE were identical in both li-pilo-DZP and li-pilo-TC groups. Within 5 min after pilocarpine injection, rats developed diarrhea, piloerection and other signs of cholinergic stimulation. During the following 15-20 min, rats exhibited head bobbing, scratching, chewing and exploratory behavior. Recurrent seizures started around 15-20 min after pilocarpine administration. These seizures which associated episodes of head and bilateral forelimb myoclonus with rearing and falling progressed to SE at about 35-40 min after pilocarpine, as previously described (Turski et al., 1989; Dubé et al., 2001; André et al., 2003). The control group not subjected to SE and receiving lithium and saline was composed of 20 rats.

In the group of 57 animals devoted to cell counting at 14 days after SE, a total number of 13 rats died over the first 48 h after SE. The degree of mortality varied with the treatment: 36% (4/11) of DZP rats, 33% (4/12) of TC30 rats, 8% (1/12) of TC60 rats, 0% (0/10) of TC90 rats and 33% (4/12) of TC120 rats died. In the DZP group, the 4 rats died in the first 24 h after SE. In the group of TC30 rats, one rat died on the day of SE, one rat was dead by 24 h after SE and two rats by 48 h. In the group of TC60 rats, one rat died at 48 h after SE. In the group of TC120 rats, two rats were dead by 24 h and two by 48 h after SE.

In the group of 55 animals devoted to the study of the latency to SRS and late cell counting, the degree of mortality over the first 48 h after SE was the following: 7% (1/14) of DZP rats, 27% (3/11) of TC30 rats, 0% (0/10) of TC60 rats, 0% (0/11) of TC90 rats and 0% (0/9) of TC120 rats died. In the group of DZP rats, one rat died during the first 24 h after SE. In the group of TC30, two rats were dead by 24 h and one by 48 h after SE.

Cell Densities in Hippocampus and Cortex in the Early Phase (14 Days After SE)

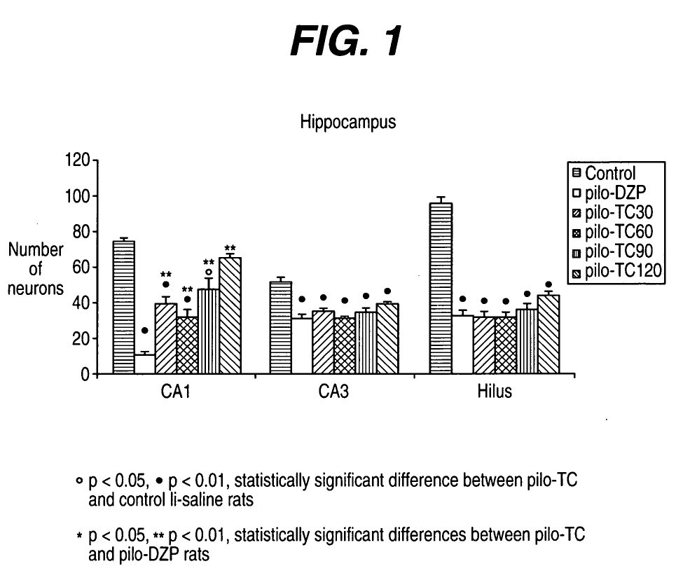

In DZP rats compared to control rats, the number of neurons was massively decreased in the CA1 region of the hippocampus (85% drop out in the pyramidal cell layer) while the CA3 region was less extensively damaged (40% loss) (Table 1 and FIG. 1). In the dentate gyrus, DZP rats experienced extensive neuronal loss in the hilus (65%) while the granule cell layer did not show overt damage. The same distribution of damage was observed in the ventral hippocampus but cell counts were not performed in this region.

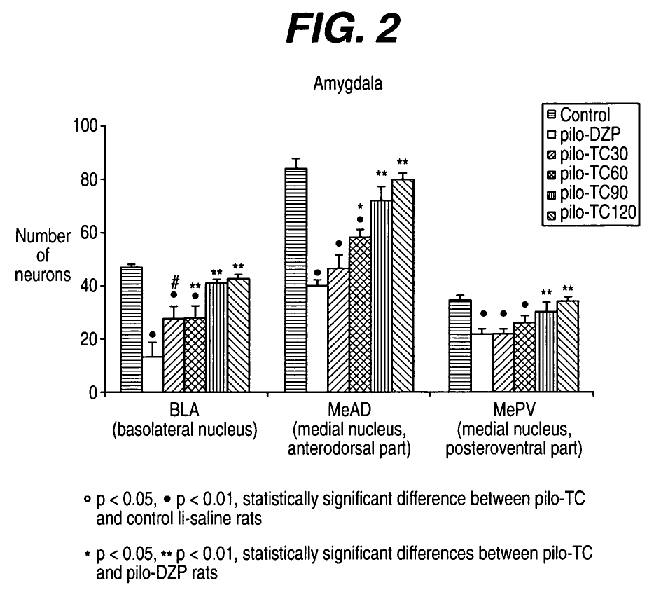

In the thalamus, neuronal loss was moderate in the mediodorsal central and lateral, the dorsolateral medial dorsal and in the central medial nuclei (18, 24, 40 and 34% drop out, respectively), more marked in the mediodorsal nucleus (49%) and major in the ventral lateral division of the dorsolateral nucleus (90%) (Table 1 below and FIG. 2).

TABLE 1
Effects of increasing doses of TC on the number of neuronal cell bodies in the hippocampus,
thalamus, amygdala and cerebral cortex of rats subjected to li-pilo SE.

| | Control | pilo-DZP | pilo-TC30 | pilo-TC60 | pilo-TC90 | pilo-TC120 |
|---|---|---|---|---|---|---|
| | (n = 10) | (n = 7) | (n = 8) | (n = 11) | (n = 10) | (n = 8) |
| Hippocampus | | | | | | |
| CA1 area | 74.8 ± 1.5 | 10.9 ± 1.9** | 39.3 ± 4.4**°° | 31.9 ± 4.4**°° | 47.7 ± 6.6*° | 65.5 ± 2.9°° |
| CA3 area | 52.1 ± 2.7 | 31.3 ± 2.9** | 35.7 ± 1.8** | 31.6 ± 1.4** | 35.1 ± 2.9** | 39.8 ± 1.5** |
| Hilus | 96.4 ± 3.5 | 33.5 ± 3.0** | 33.0 ± 3.2** | 32.8 ± 3.3** | 37.5 ± 3.1** | 44.8 ± 2.9** |
| Thalamus | | | | | | |
| Mediodorsal medial | 31.9 ± 0.9 | 16.4 ± 1.9** | 11.5 ± 2.5** | 19.1 ± 2.6** | 23.1 ± 2.8°° | 28.6 ± 0.8°° |
| Mediodorsal central | 31.9 ± 1.2 | 26.3 ± 1.8** | 26.9 ± 0.6* | 24.1 ± 1** | 27.4 ± 1.5 | 29.9 ± 1.7° |
| Mediodorsal lateral | 25.9 ± 0.6 | 19.6 ± 0.8** | 20.5 ± 0.7** | 18.9 ± 0.6** | 22 ± 1.2*° | 24.4 ± 1.1°° |
| Dorsolateral, medial, dorsal | 102.2 ± 2.5 | 61 ± 6.3** | 64.2 ± 9.3**°° | 77.5 ± 3.9**°° | 79.4 ± 3.1**°° | 89.8 ± 3.7*° |
| Dorsolateral, ventral lateral | 97.8 ± 1.7 | 9.7 ± 2.5** | 8.8 ± 2.8** | 56.7 ± 8.7** | 71.8 ± 5.3°°* | 79.0 ± 4.7°° |
| Central medial | 113.1 ± 5.9 | 74.2 ± 7.4* | 75.6 ± 7.7* | 83.7 ± 9.6* | 88.2 ± 8.5 | 108.2 ± 6.6° |
| Amygdala | | | | | | |
| Basolateral | 46.7 ± 1.2 | 12.8 ± 5.3** | 27.3 ± 4.9**° | 27.8 ± 4.3**°° | 40.7 ± 1.6°° | 42.7 ± 1.3°° |
| Medial, dorsal anterior | 84.3 ± 3.8 | 40.0 ± 2.5** | 46.8 ± 5.0** | 58.4 ± 2.8**° | 72.2 ± 5.7°° | 80.2 ± 2.6°° |
| Medial, ventral posterior | 35.1 ± 1.7 | 21.8 ± 2.4** | 22.3 ± 1.8** | 26.2 ± 2.9** | 30.7 ± 3.7°° | 34.7 ± 1.7°° |
| Cerebral cortex | | | | | | |
| Piriform, layer II, dorsal | 36.6 ± 0.8 | 12.6 ± 4.2** | 15.7 ± 2.9** | 27.5 ± 2.8**°° | 32.4 ± 1.1°° | 35.2 ± 1.1°° |
| Piriform, layer II, ventral | 33.0 ± 0.8 | 3.6 ± 0.7** | 7.2 ± 3.8** | 13.7 ± 4.2** | 18.4 ± 4.0°° | 30.5 ± 1.3°° |
| Piriform, layer III | 19.2 ± 0.7 | 1.2 ± 1.2** | 1.8 ± 1.8** | 6.4 ± 2.3** | 9 ± 3.0°° | 15 ± 2.2°° |
| Entorhinal, layer II, dorsal | 29 ± 0.6 | 23.5 ± 0.7** | 23.4 ± 0.6** | 23.9 ± 0.5** | 26.3 ± 0.9** | 27.3 ± 0.5°° |
| Entorhinal, layer II, ventral | 26.8 ± 0.7 | 21.7 ± 1.3** | 22.7 ± 0.9 | 23.3 ± 0.8** | 25.4 ± 1.1° | 25.1 ± 0.6 |
| Entorhinal, layer III/IV, | 29.2 ± 0.9 | 22.3 ± 0.5** | 22.3 ± 0.5** | 23.2 ± 0.8** | 26.7 ± 0.8* | 26.4 ± 0.7°° |
| dorsal | | | | | | |
| Entorhinal, layer III/IV, | 28.7 ± 1.7 | 7.7 ± 2.3** | 13.2 ± 1.9** | 16.5 ± 2.2** | 23.7 ±1.5°° | 24.5 ± 1.4°° |
| ventral | | | | | | |

*p < 0.05,
**p < 0.01, statistically significant difference between pilo-TC and control li-saline rats
°p < 0.05,
°°p < 0.01, statistically significant differences between pilo-TC and pilo-DZP rats

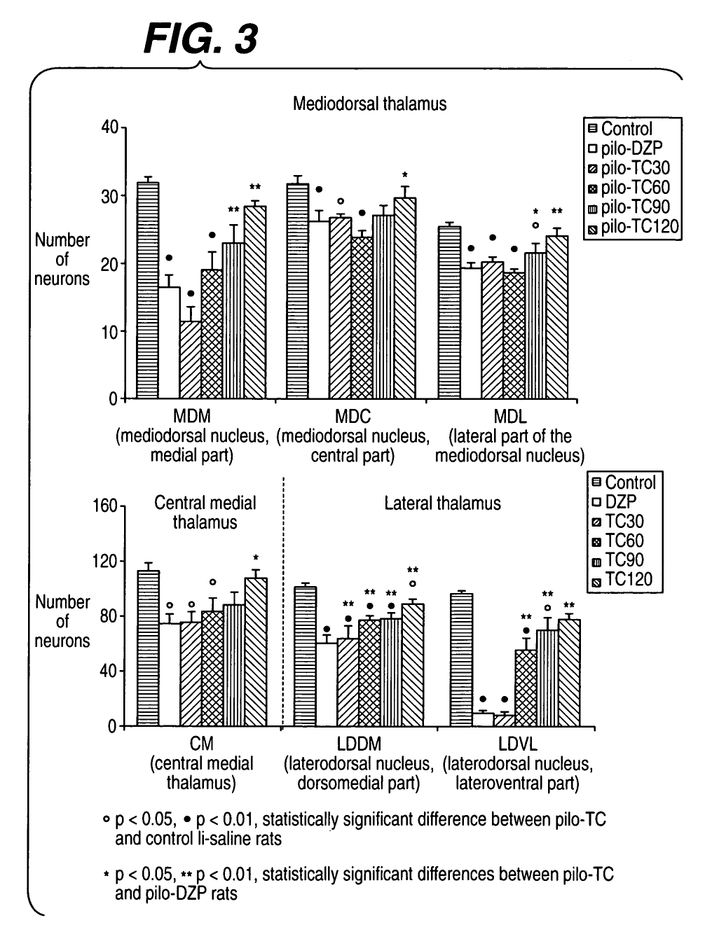

In the amygdala, neuronal loss was moderate in the medial ventral posterior nucleus (38%) and more marked in the basolateral and medial dorsal anterior nuclei (73 and 53% drop out, respectively). There was no neuronal damage in the central nucleus (Table 1 and FIG. 3).

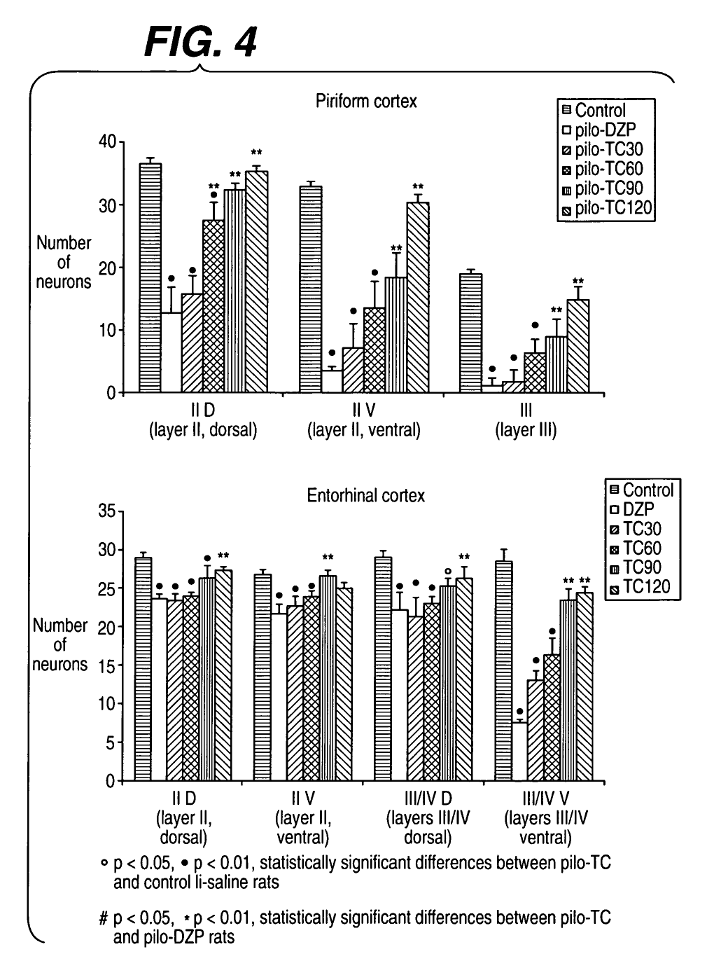

In the piriform cortex, neuronal loss was almost total in layer III (94%) which was no longer really visible and reached 66 and 89% in dorsal and ventral layer II, respectively in DZP rats compared to control saline-treated rats. In the dorsal entorhinal cortex, layers II and III-IV underwent slight damage (18 and 24%, respectively) and in ventral layers II and III/IV, damage reached 22 and 74%, respectively (Table 1 and FIG. 4).

In the hippocampus of TC-treated animals, cell loss was significantly reduced compared to DZP rats in CA1 pyramidal cell layer. This reduction was marked in TC30 , 60 or 90 rats (36-47% cell loss) and prominent in the TC120 group (12% cell loss). The differences were statistically significant at all TC doses (Table 1 and FIG. 1). In the CA3 pyramidal layer, there was a tendency to a slight neuroprotection induced by RWJ, only at the 120 mg/kg dose but the difference with the DZP group was not significant. In the dentate gyrus, the cell loss in the hilus was similar in the DZP and TC30, 60 and 90 groups (61-66% drop out) and there was a slight tendency to reduced damage in the TC1 20 group (53% neuronal loss) compared to DZP animals (66% drop out). None of these differences was statistically significant.

In the thalamus, neuronal loss was similar in DZP and TC30 and TC60 rats. TC was significantly protective at the 60 mg/kg dose in the dorsolateral medial dorsal nucleus and at the two highest doses, 90 and 120 mg/kg in all thalamic nuclei, although the difference did not reach significance in the mediodorsal central and central medial nuclei in TC90 rats. In TC120 rats, neuronal drop out was considerably reduced compared to DZP rats. It ranged from 4-19% and the number of neurons was no longer significantly different from control animals, except in the dorsolateral medial dorsal nucleus (Table 1 and FIG. 2). In the amygdala, TC was significantly protective at the 30 mg/kg dose in the basolateral nucleus and at the 60 mg dose, also in the medial dorsal anterior nucleus. At the highest dose, TC was largely neuroprotective; the number of neurons was no longer significantly different from the control level and reached 86-99% of the control level in all amygdala nuclei (Table 1 and FIG. 3).

In the cerebral cortex, the treatment with TC did not significantly protect any cortical area compared to the DZP treatment at the dose of 30 mg/kg. At 60 mg/kg, TC significantly reduced neuronal loss only in layer II of the dorsal piriform cortex (25% drop out compared to 66% in the DZP group). At 90 and 120 mg/kg, TC significantly protected all three areas of the piriform cortex compared to the DZP treatment and at the highest dose of TC, 120 mg/kg, neuronal density reached 78-96% of control levels, even in piriform cortex, dorsal layer II and layer III where the neuronal population was almost totally depleted in the DZP group. In all layers of the dorsal and ventral entorhinal cortex, the two lowest doses of TC, 30 and 60 mg/kg did not afford any neuroprotection. The 90-mg/kg dose of TC significantly protected layers II and III/IV of the ventral entorhinal cortex (4 and 17% damage remaining in layers II and II/IV of the dorsal part and in layer II of the ventral part compared to 19 and 73% in the DZP group). At the highest dose of TC, 120 mg/kg, all parts of the entorhinal cortex, both dorsal and ventral were protected and the number of neurons in these areas was no longer significantly different from the level in controls (85-94% of neurons surviving compared to 27-81% in the DZP group). Latency to and frequency of recurrent seizures

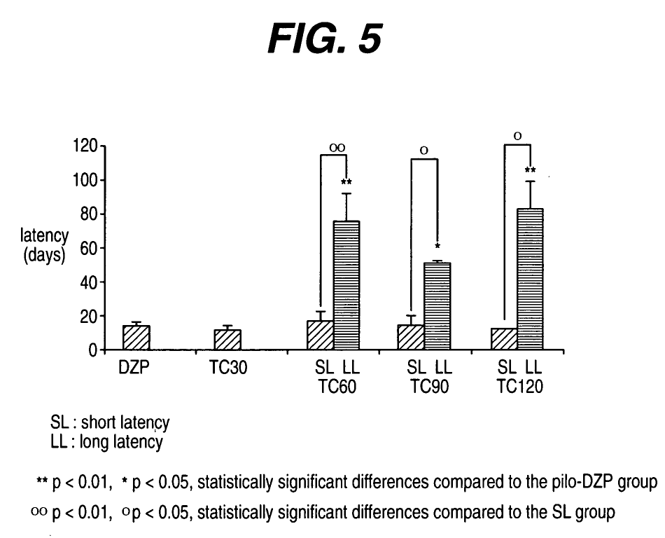

The latency to spontaneous seizures reached a mean value of 15.5±2.3 days in the DZP group (14 rats) and was similar (11.6±2.5 days) in the TC30 group (8 rats). At higher concentrations of TC, animals could be subdivided in subgroups with short and long latencies. A short latency was considered as any duration shorter than 40 days after SE. Some rats exhibited a latency to the first spontaneous seizure that was similar to that recorded in the DZP and TC groups but the number of rats exhibiting this short latency values progressively decreased with the increase in TC concentration. Thus at 30 mg/kg, 70% of the rats (7/10) had short latencies to seizures while at 90 and 120 mg/kg, this percentage reached 36% (4/11) and 11% (1/9), respectively (Table 2 below and FIG. 5).

TABLE 2
Effect of increasing doses of TC on the latency to spontaneous seizures.

| | Number of | Latency to the first spontaneous seizure | |
|---|---|---|---|
| Treatment | animals | (days) | |
| DZP | 14 | 15.5 ± 2.34 | |
| pilo-TC30 | 8 | 11.6 ± 2.5 | |
| | | 2 groups | |
| pilo-TC60 | 10 | Short latency (n = 7) | Long latency (n = 3) |
| | | 17.4 ± 5.4 | 76.7 ± 15.6** °° |

| | | 3 groups | | |
|---|---|---|---|---|
| pilo-TC90 | 11 | Short latency (n = 4) | Long latency (n = 2) | Non epileptic (n = 5) |
| | | 14.8 ± 5.7 | 52.0 ± 1.0* ° | 150** °° |
| | | 3 groups | | |
| pilo-TC120 | 9 | Short latency (n = 1) | Long latency (n = 4) | Non epileptic (n = 4) |
| | | 13.0 | 84.5 ± 16.7** °° | 150** °° |

**p < 0.01,
*p < 0.05, statistically significant differences compared to the pilo-DZP group
°° p < 0.01,
° p < 0.05, statistically significant differences compared to the short latency group

In the TC60, 90 and 120 groups, the mean value of the rats with long latencies was similar and ranged from 52 to 85 days. Finally, at the two highest doses of TC, we were able to identify a percentage of rats that did not develop any seizure over a duration of 150 days post-SE. The percentage of non-epileptic rats reached 45% at both doses of TC.

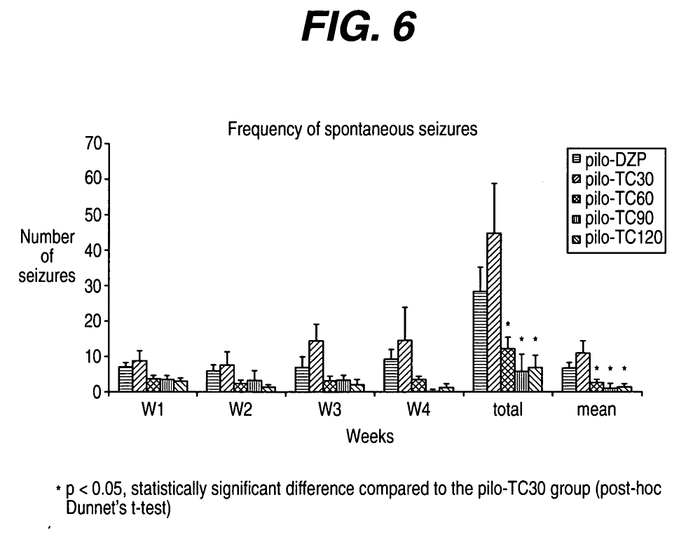

The frequency of spontaneous seizures was similar over the four weeks of recording. It showed a tendency to be higher in the DZP and TC30 groups while it was lower in the TC60, 90 and 120 groups (FIG. 6). These differences did not reach statistical significance at the level of each individual weekly frequency but reached significance for the total or mean number of seizures over the four weeks.

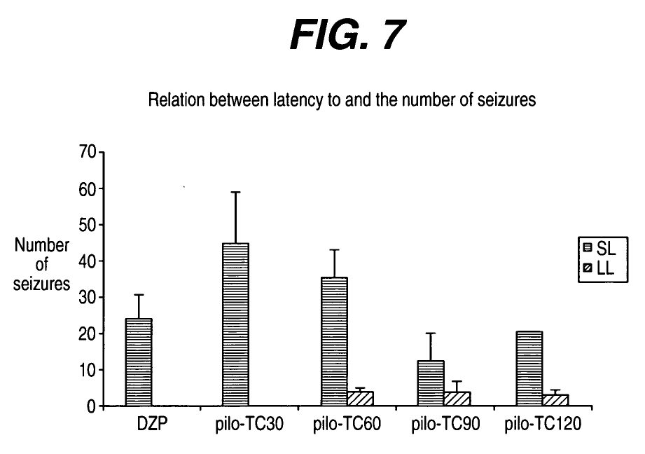

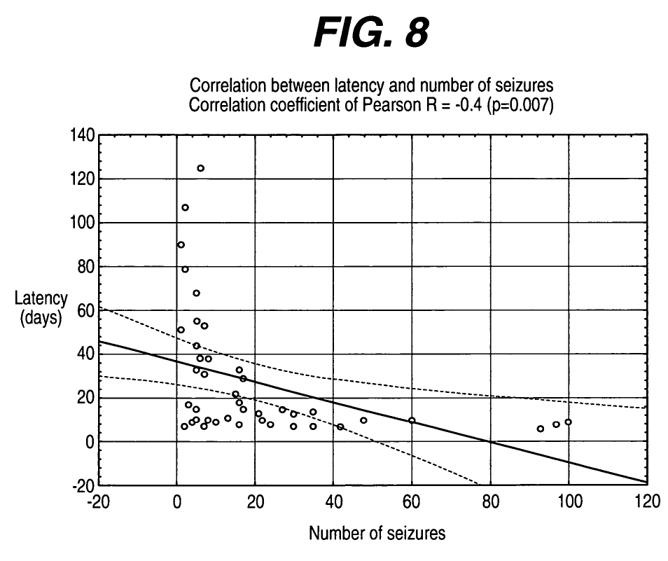

The number of seizures was also plotted according to the duration of the latency to the first spontaneous seizure. Animals with a short latency showed a tendency to display 2-3 times more seizures over the four weeks of recording than rats with a long latency period. No statistical analysis could be performed since the ANOVA did not show any significance, most likely because there was only one animal in the short latency subgroup of the TC120 animals (FIG. 7). However, when all latency values were plotted against the number of seizures, there was a significant inverse correlation leading to a straight line with a correlation coefficient of −0.4 (FIG. 8).

To finalize this analysis, we need to perform two more measurements. The first one is cell counting on the animals that were video recorded and followed for 2 months after the first spontaneous seizure or sacrificed at 5 months to study the potential correlation between the extent and location of brain damage and the occurrence of and/or latency to spontaneous seizures. The second one will be to perform a one year follow-up of seizure occurrence in a group of rats to study whether or not the animals that we declare “non epileptic” at 5 months will remain seizure free.

The results of the present study show that a treatment with TC starting at 1 h after the onset of Li-pilo-induced SE has neuroprotective properties in the CA1 pyramidal cell layer of the hippocampus, and in all layers of the ventral and dorsal piriform and entorhinal cortex. TC protects also thalamus and amygdala nuclei. However, TC is not protective at the dose of 30 mg/kg, except in CA1, one thalamic and one amygdala nucleus. At the dose of 60 mg/kg, layer II of the dorsal piriform cortex and a second amygdala nucleus are also protected. At 90 and 120 mg/kg, the drug protects most cerebral regions studied, except hippocampal CA3 and the hilus of the dentate gyrus. The latter two structures plus the dorsolateral ventral dorsal thalamic nucleus are the only regions where the number of neurons remains significantly different from controls at the dose of 120 mg/kg TC. From these data, the extremely powerful neuroprotection properties of TC appear clearly. The molecule seems to prevent neuronal death in most regions belonging to the circuit of limbic epilepsy induced by Li-pilo, i.e., the hippocampus, thalamus, amygdale and parahippocampal cortices. These are all the regions in which we have detected MRI signal in the course of epileptogenesis in Li-pilo-treated rats (Roch et al., 2002a). The only two regions that are not efficiently protected by TC are CA3 pyramidal cell layer and the hilus of the dentate gyrus. The latter region undergoes rapid and massive cell damage (André et al., 2001; Roch et al., 2002a) and none of the neuroprotection that we used in previous studies have been able to protect this structure. On the basis of earlier studies identified this structure has been identified as a key area in the initiation and maintenance of epileptic seizures in the Li-pilo model (Dubé et al., 2000). Obviously, the present data demonstrate that epileptogenesis can be prevented even though damage remains quite marked in this area. Long-term cell counting on the group of animals that has been video recorded will be able to show whether or not the extent of damage in this region is critical for epileptogenesis in this model.

The treatment did not affect the latency to the first spontaneous seizure at the dose of 30 mg/kg. At the 3 higher doses, a percentage of animals developed epilepsy as fast as the DZP or TC30 rats but the relative importance of this subgroup was inversely related to the dose of TC used. Another subgroup, constant in size (2-4 animals per group) developed epilepsy after a 4-6 times longer latency while at the two highest doses of the drug, 4-5 rats had not become epileptic after 5 months, i.e. about 10 times the duration of the short latency and 2-3 times that of the long latency. This delay in the occurrence of epilepsy might correlate with the number of neurons protected in the basal cortices in the animals. This assumption is based on the fact that we noted some heterogeneity in the extent of neuroprotection in basal cortices of the animals subjected the short term neuronal counting at 14 days after SE. However, at the moment, we have not performed neuronal counting in the animals used for the study of epileptogenesis and therefore, no conclusion can be drawn on a potential relation between the number of neurons surviving in basal cortices and the rate or even occurrence of epileptogenesis.

The data obtained in the present study are in line with the previous study from our group reporting that the 60-mg/kg dose of TC protected the hippocampus and the basal cortices from neuronal damage and delayed the occurrence of recurrent seizures (see previous report, 2002). They confirm that the protection of the basal cortices could be a key factor in inducing a disease modifying effect in the lithium-pilocarpine model of epilepsy. The key role of the basal cortices as initiators of the epileptic process was previously demonstrated by our group in the lithium-pilocarpine model (André et al., 2003; Roch et al., 2002a,b).

In conclusion, the results of this study shows that the test compound (TC) has very promising anti-epileptogenic effects.

References for Example 2

André V, Marescaux C, Nehlig A, Fritschy J M (2001) Alterations of the hippocampal GABAergic system contribute to the development of spontaneous recurrent seizures in the lithium-pilocarpine model of temporal lobe epilepsy. Hippocampus 11:452-468.
André V, Rigoulot M A, Koning E, Ferrandon A, Nehlig A (2003) Long-term pregabalin treatment protects basal cortices and delays the occurrence of spontaneous seizures in the lithium-pilocarpine model in the rat. Epilepsia 44:893-903.
Cavalheiro E A (1995) The pilocarpine model of epilepsy. Ital J Neurol Sci 16:33-37.
Dubé C, Marescaux C, Nehlig A (2000) A metabolic and neuropathological approach to the understanding of plastic changes occurring in the immature and adult rat brain during lithium-pilocarpine induced epileptogenesis. Epilepsia 41 (Suppl 6):S36-S43.
Dubé C, Boyet S, Marescaux C, Nehlig A (2001) Relationship between neuronal loss and interictal glucose metabolism during the chronic phase of the lithium-pilocarpine model of epilepsy in the immature and adult rat. Exp Neurol 167:227-241.
Paxinos G, Watson C (1986) The Rat Brain in Stereotaxic Coordinates, 2nd ed. Academic Press, San Diego.
Roch C, Leroy C, Nehlig A, Namer I J (2002a) Contribution of magnetic resonance imaging to the study of the lithium-pilocarpine model of temporal lobe epilepsy in adult rats. Epilepsia 43:325-335.
Roch C, Leroy C, Nehlig A, Namer I J (2002b) Predictive value of cortical injury for the development of temporal lobe epilepsy in P21-day-old rats: a MRI approach using the lithium-pilocarpine model. Epilepsia 43:1129-1136.
Turski L, Ikonomidou C, Turski W A, Bortolotto Z A, Cavalheiro E A (1989) Review: Cholinergic mechanisms and epileptogenesis. The seizures induced by pilocarpine: a novel experimental model of intractable epilepsy. Synapse 3:154-171.

### Example 3

PC12 Cell Serum Withdrawal Model

Serum withdrawal is a cytotoxic environmental challenge that results in cell death in cultured cell lines as well as in primary cells of various tissue origins, including nerve cells. In particular, pheochromocytoma (PC) 12 cells have been widely employed as an in vitro neuronal cell model for a wide variety of neurodegenerative and cell death related disorders (Muriel, et al, Mitochondrial free calcium levels (Rhod-2 fluorescence) and ultrastructural alterations in neuronally differentiated PC12 cells during ceramide-dependent cell death, J. Comp. Neurol., 2000, 426(2), 297-315; Dermitzaki, et al, Opioids transiently prevent activation of apoptotic mechanisms following short periods of serum withdrawal, J. Neurochem., 2000, 74(3), 960-969; Carlile, et al, Reduced apoptosis after nerve growth factor and serum withdrawal: conversion of tetrameric glyceraldehyde-3-phosphate dehydrogenase to a dimer, Mol Pharmacol., 2000, 57(1), 2-12). PC12 cells were cultured in sterile media (RPMI 1640) supplemented with 10% heat-inactivated horse serum and 5% fetal bovine serum (FBS). The culture medium also contained Penicillin-Streptomycin-Neomycin antibiotic (50 .mu.g, 50 .mu.g, 100 .mu.g, respectively). Medium was exchanged every other day and the cells were passed in log phase near confluence.

The control cells were cultured in regular media without any treatment. An enantiomer of Formula 7 or Formula 8 (10 .mu.M) was mixed well in the medium and then applied to the cells. For the 2 day assay, an enantiomer of Formula 7 or Formula 8 (10 .mu.M) was only applied to the cells once at the time of serum withdrawal. For the 7 day assay, an enantiomer of Formula 7 or Formula 8 (10. mu.M) was applied to the cells at the time of serum withdrawal and every 48 hr thereafter when cells were changed with fresh new serum-free medium. In the serum withdrawal group, the cells were cultured in serum-free medium with no additional enantiomer of Formula 7 or Formula 8. Cell survival was determined by the 3-(4,5-dimethylthiazol-2-yl)-5-(3-carboxy-methoxyphenyl)-2-(4-sulfophenyl)-2H-tetrazolium inner salt (MTS) assay at 2 or 7 days after serum withdrawal.

At the end of the experiment, cells were washed with fresh medium and incubated with MTS solution in a humidified 37.degree. C. with 5% CO2 incubator for 1.5 hr. After the incubation period, the cells were immediately analyzed using a Softmax program (Molecular Devices). MTS assay is a calorimetric method for determining the number of viable cells in a given experimental setting. The assay is based on the cellular conversion of the tetrazolium salt, MTS, into a formazan that is soluble in tissue culture medium and measured directly at 490 nm in 96-well assay plates. The absorbance is directly proportional to the number of living cells in culture. The arbitrary absorbance reading in control cells is expressed as 100% survival rate.

Table 3 lists data demonstrating the effect on cell survival rate of the orally administered enantiomer of Formula 7 and Formula 8 in the PC12 cell serum withdrawal model.

TABLE 3
(% CELL SURVIVAL RATE)

| | 2 Day Survival | 7 Day Survival |
|---|---|---|
| | Rate (%) | Rate (%) |
| Control | 100 | 100 |
| Serum-free | 49.6 ± 2.6 | 23.8 ± 2.6 |
| Formula 7 | 69.4 ± 1.7¹ | 79.9 ± 4.0² |
| Formula 8 | 66.4 ± 5.4¹ | 85.2 ± 0.6² |

### Example 4

The Transient Cerebral Ischemia Rat Model

The enantiomer of Formula 7 (test compound) was investigated in the transient cerebral ischemia middle cerebral artery occlusion (MCAO) rat model (as described in Nagasawa H. and Kogure K., Stroke, 1989, 20, 1037; and, Zea Longa E., Weinstein P. R., Carlson S. and Cummins R., Stroke, 1989, 20, 84) using male Wistar rats at 10 and 100 mg/kg (i.v.). MK 801 (Dizocilpine maleate; CAS Registry number 77086-22-7, a commercially available neuroprotectant compound) was used as a positive control (3 mg/kg, i.p.).

Rats (n=12) were randomly allocated to one of four experimental groups and were anesthetized. Blood flow from the internal carotid artery, anterior cerebral artery and posterior cerebral artery into the middle cerebral artery was blocked by this procedure. One hour after blockage, animals were treated over a 1 hour period with vehicle (administered i.v. over the one hour period), control (administered as a single i.p. dose at the start of the one hour period) and two doses of the enantiomer of Formula 7 (administered i.v. over the one hour period). Two hours after blockage, reperfusion was performed.

The animals were sacrificed and 20 mm-thick coronal sections of each brain were prepared. One in every forty sections (i.e. every 800 nM) from the front to the occipital cortex was used to quantify the extent of the cerebral lesion. Slides were prepared using sections stained (according to the Nissl procedure) with cresyl violet and were examined under a light microscope.

Regional ischemic surface areas in the coronal sections of individual rats were determined according to the presence of cells with morphological changes. The areas of neuronal injury or infarction were measured and then added. The cortex and striatum volume were calculated for each animal (total ischemic surface area.times.0.8 mm (thickness)).

MCAO Model Analysis

The mean volumes (.+−.S.E.M.) for each animal randomly assigned to the four experimental groups were compared using one-way ANOVA (one way ANOVA is a statistical method which compares 3 or more unmatched groups) followed by Dunnett's t-test (both methods incorporated in Statview 512+software, BarinPower, Calabasas, Calif., USA).

As shown in Table 4 below, results were considered statistically significant when the p value was <0.05 compared to vehicle group (¹p<0.01; ²p<0.05).

TABLE 4

| | | Mean Infarct Volume (mm³) ± S.E.M. | | |
|---|---|---|---|---|
| | | Cortex | | |
| Treatment | N | Volume | Striatum | Total |
| Vehicle, 10 mL/kg | 12 | 275.5 ± 27.1 | 79.4 ± 3.6 | 354.9 ± 29.9 |
| MK 801 @ 3 mg/kg | 12 | 95.8 ± 24.5¹ | 56.1 ± 5.3² | 151.9 ± 28.7¹ |
| Formula 7 @ 10 | 12 | 201.0 ± 23.9 | 75.9 ± 2.6 | 276.9 ± 25.4 |
| mg/kg | | | | |
| Formula 7 @ 100 | 12 | 98.8 ± 29.5¹ | 63.0 ± 5.9² | 161.9 ± 34.3¹ |
| mg/kg | | | | |

References Cited

All references cited herein are incorporated herein by reference in their entirety and for all purposes to the same extent as if each individual publication or patent or patent application was specifically and individually indicated to be incorporated by reference in its entirety for all purposes.

The discussion of references herein is intended merely to summarize the assertions made by their authors and no admission is made that any reference constitutes prior art. Applicants reserve the right to challenge the accuracy and pertinence of the cited references.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

What is claimed is:

1. A method for providing neuroprotection, comprising administering to a patient in need of treatment with a neuroprotective drug (an NPD) a therapeutically effective amount of a compound, or a pharmaceutically acceptable salt or ester thereof, selected from the group consisting of Formula (I) and Formula (II): 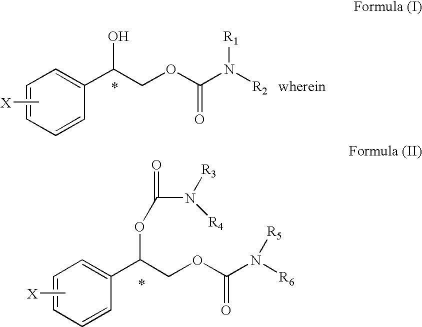
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

2. The method of claim 1 wherein X is chlorine.

3. The method of claim 1 wherein X is substituted at the ortho position of the phenyl ring.

4. The method of claim 1 wherein R₁, R₂, R₃, R₄, R₅ and R₆ are selected from hydrogen.

5. A method for providing neuroprotection, comprising administering to a patient in need of treatment with a neuroprotective drug (an NPD) a therapeutically effective amount of an enantiomer, or a pharmaceutically acceptable salt or ester thereof, selected from the group consisting of Formula (I) and Formula (II) or enantiomeric mixture wherein one enantiomer selected from the group consisting of Formula (I) and Formula (II) predominates: 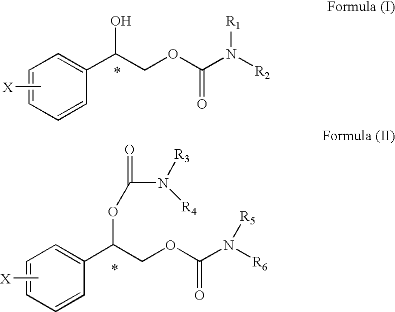
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

6. The method of claim 5 wherein X is chlorine.

7. The method of claim 5 wherein X is substituted at the ortho position of the phenyl ring.

8. The method of claim 5 wherein R₁, R₂, R₃, R₄, R₅ and R₆ are selected from hydrogen.

9. The method of claim 5 wherein one enantiomer selected from the group consisting of Formula (I) and Formula (II) predominates to the extent of about 90% or greater.

10. The method of claim 5 wherein one enantiomer selected from the group consisting of Formula (I) and Formula (II) predominates to the extent of about 98% or greater.

11. The method of claim 5 wherein the enantiomer selected from the group consisting of Formula (I) and Formula (II) is an enantiomer selected from the group consisting of Formula (Ia) and Formula (IIa): 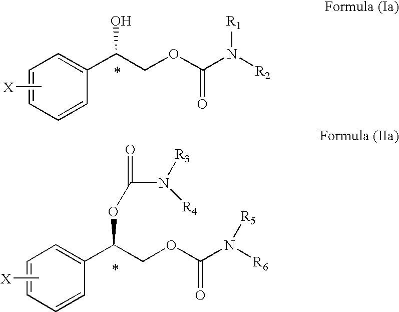
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano).

12. The method of claim 11 wherein X is chlorine.

13. The method of claim 11 wherein X is substituted at the ortho position of the phenyl ring.

14. The method of claim 11 wherein R₁, R₂, R₃, R₄, R₅ and R₆ are selected from hydrogen.

15. The method of claim 11 wherein one enantiomer selected from the group consisting of Formula (Ia) and Formula (IIa) predominates to the extent of about 90% or greater.

16. The method of claim 11 wherein one enantiomer selected from the group consisting of Formula (Ia) and Formula (IIa) predominates to the extent of about 98% or greater.

17. The method of claim 5 wherein the enantiomer selected from the group consisting of Formula (I) and Formula (II) is an enantiomer selected from the group consisting of Formula (Ib) and Formula (IIb):

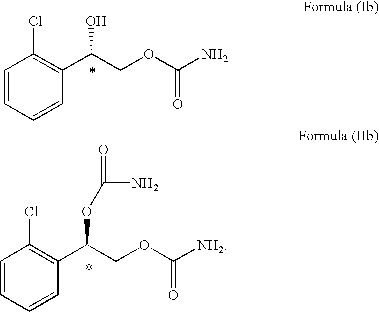

18. The method of claim 17 wherein one enantiomer selected from the group consisting of Formula (Ib) and Formula (IIb) predominates to the extent of about 90% or greater.

19. The method of claim 17 wherein one enantiomer selected from the group consisting of Formula (Ib) and Formula (IIb) predominates to the extent of about 98% or greater.

20. The method, as claimed in claims 1 or 5 wherein the possible cause(s) of neuronal damage rendering the patient in need of neuroprotection are selected from the group consisting of: Traumatic Brain Injury (TBI), injury or trauma of any kind to the CNS or PNS including blunt and penetrating head trauma; infections of the CNS; anoxia; stroke (CVAs); autoimmune diseases affecting the CNS, e.g., lupus; birth injures, e.g., perinatal asphyxia; cardiac arrest; therapeutic or diagnostic vascular surgical procedures, e.g., carotid endarterectomy or cerebral angiography; spinal cord trauma; hypotension; injury to the CNS from emboli, hyper or hypo perfusion; metabolic disorders, e.g., diabetes, hypoxia; known genetic predisposition to disorders known to respond to NPDs; space occupying lesions of the CNS; brain tumors, e.g., glioblastomas; bleeding or hemorrhage in or surrounding the CNS, e.g., intracerebral bleeds or subdural hematomas; brain edema; febrile convulsions; hyperthermia; substance abuse, trauma, stroke, ischemia, Huntington's disease, Alzheimer's disease, Parkinson's disease, prion disease variant Creutzfeld-Jakob disease, amyotrophic lateral sclerosis (ALS), diabetic neuropathy, olivopontocerebellar atrophy, epilepsy, seizures, hypoglycemia, surgery or other interventions, retinal ischemia (diabetic or otherwise), glaucoma, retinal degeneration, multiple sclerosis, toxic and ischemic optic neuropathy, macular degeneration, exposure of the CNS or PNS to toxic or poisonous agents; drug intoxication or withdrawal, e.g. cocaine or alcohol; family history of; neurodegenerative disorders or a related condition, history of status epilepticus; evidence from surrogate markers or biomarkers that the patient is in need of treatment with a neuroprotective drug (NPD), e.g., MRI scan showing structural or functional pathology, elevated serum levels of neuronal degradation products, elevated levels of ciliary neurotrophic factor (CNTF).

21. The method of claim 20 wherein the predisposing factor(s) rendering the patients in need of neuroprotection are selected from the group consisting: Traumatic Brain Injury (TBI), blunt, closed and penetrating head trauma; surgery, stroke or other cerebral-vascular accident (CVA); status epilepticus and space occupying lesions of the CNS.

22. The method of claim 21 wherein the said predisposing factor(s) are Traumatic Brain Injury (TBI) including blunt, closed or penetrating head trauma and surgical intervention.

23. The method of claim 21 wherein the said predisposing factor(s) are stroke or other cerebral-vascular accident (CVA).

24. The method of claim 23 wherein the said predisposing factor is a neurodegenerative disease.

25. The methods of claims 1 or 5 wherein said compound (or enantiomer) or a pharmaceutically acceptable salt or ester thereof is administered in combination administration with one or more other compounds or therapeutic agents.

26. The methods of claim 25 wherein the said one or more other compounds or therapeutic agents are selected from the group consisting of compounds that have one or more of the following properties: antioxidant activity; NMDA receptor antagonism; ability to augment endogenous GABA inhibition; NO synthase inhibitor activity; iron binding ability, e.g., an iron chelator; calcium binding ability, e.g., a Ca (II) chelator; zinc binding ability, e.g., a Zn (II) chelator; the ability to block sodium or calcium ion channels; the ability to open potassium or chloride ion channels; such that neuroprotective effects are provided to the patient.

27. The methods of claim 26 wherein the said one or more compounds may, in addition, be selected from the group consisting of anti-epileptic drugs (AEDs).

28. The methods of claim 27 wherein the said anti-epileptic drug (AED) is selected from the group consisting of; carbamazepine, clobazam, clonazepam, ethosuximide, felbamate, gabapentin, lamotigine, levetiracetam, oxcarbazepine, phenobarbital, phenytoin, pregabalin, primidone, retigabine, talampanel, tiagabine, topiramate, valproate, vigabatrin, zonisamide, benzodiazepines, barbiturates or a sedative hypnotic.

29. A pharmaceutical composition for providing neuroprotection comprising a pharmaceutically effective amount of an enantiomer, or a pharmaceutically acceptable salt or ester thereof, selected from the group consisting of Formula (I) and Formula (II) or enantiomeric mixture wherein one enantiomer selected from the group consisting of Formula (I) and Formula (II) predominates: 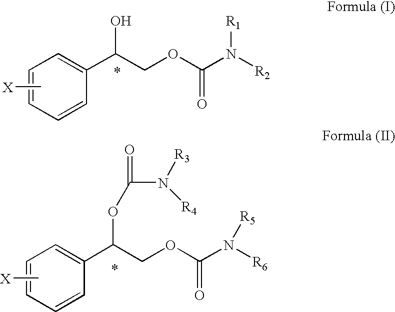
wherein
phenyl is substituted at X with one to five halogen atoms selected from the group consisting of fluorine, chlorine, bromine and iodine; and,
R₁, R₂, R₃, R₄, R₅ and R₆ are independently selected from the group consisting of hydrogen and C₁-C₄ alkyl; wherein C₁-C₄ alkyl is optionally substituted with phenyl (wherein phenyl is optionally substituted with substituents independently selected from the group consisting of halogen, C₁-C₄ alkyl, C₁-C₄ alkoxy, amino, nitro and cyano) and a pharmaceutically acceptable carrier or excipient.

30. A kit, comprising therapeutically effective dosage forms of the pharmaceutical composition claimed in claim 29 in an appropriate package or container together with information or instructions for proper use thereof to provide neuroprotection to a patient in need thereof.

31. The method as in claims 1 or 5 wherein the therapeutically effective amount is from about 0.01 mg/Kg/dose to about 100 mg/Kg/dose.

32. The method, as claimed in claims 1 or 5, wherein said patient has not developed clinical signs or symptoms of neuronal injury or dysfunction at the time of said administration.

33. The method, as claimed in claims 1 or 5, wherein said patient is at risk for developing neuronal injury or dysfunction at the time of said administration.

34. The method, as claimed in claims 1 or 5, wherein said patient has developed a neurodegenerative disorder or clinical evidence of neuronal injury at the time of said administration.
